(19) United States
(12) Patent Application Publication
(10) Pub. No.: US 2026/0334904 A1
(43) Pub. Date: Oct. 29, 2026

(54) METHODS AND SYSTEMS FOR PREDICTING RISK OF VERTEBRAE FRACTURE

(71) Applicants: Texas Medical Center, Houston, TX (US); Baylor College of Medicine, Houston, TX (US); The University of Texas MD Anderson Cancer Center, Houston, TX (US)

(72) Inventors: Niki Shakouri, Houston, TX (US); Alex Flores, Houston, TX (US); Alexander E. Ropper, Houston, TX (US); Vijay Nitturi, Houston, TX (US); Christopher Alvarez-Breckenridge, Houston, TX (US)

(21) Appl. No.: 19/655,353

(22) Filed: Apr. 22, 2026

Related U.S. Application Data
(60) Provisional application No. 63/793,496, filed on Apr. 23, 2025.

(51) Int. Cl. A61B 6/00; A61B 6/03; A61B 6/50; G06T 7/00

(52) U.S. Cl. CPC A61B 6/5217; A61B 6/032; A61B 6/505; G06T 7/0012; G06T 2207/10081; G06T 2207/20081; G06T 2207/30012

(57) ABSTRACT

Provided herein is a computer-implemented method for predicting risks of vertebrae fracture in a patient. A computer-implemented method may include obtaining image data of a patient. A computer-implemented method may include identifying one or more vertebrae of the image data. A computer-implemented method may include analyzing, by a machine learning model, the one or more vertebrae to predict a future risk of vertebrae fracture of the one or more vertebrae for the patient, wherein the machine learning model is trained on a cohort of a population comprising at least one radiation naïve patient and wherein the analysis comprises processing features of the one or more images, the features comprising radiomic features, wherein the radiomic features correlate to a structural integrity of one or more segments of the one or more vertebrae.

### CROSS-REFERENCE TO RELATED APPLICATIONS

This application claims priority to and the benefit of U.S. Prov. App. No. 63/793,496, filed Apr. 23, 2025, the entirety of which is incorporated by reference.

### TECHNICAL FIELD

The present disclosure relates to vertebrae. In particular, the present disclosure relates to methods and systems for predicting risk of vertebrae fracture.

### SUMMARY

Provided herein is a computer-implemented method for predicting risks of vertebrae fracture in a patient. A computer-implemented method may include obtaining image data of a patient. A computer-implemented method may include identifying one or more vertebrae of the image data. A computer-implemented method may include analyzing, by a machine learning model, the one or more vertebrae to predict a future risk of vertebrae fracture of the one or more vertebrae for the patient, wherein the machine learning model is trained on a cohort of a population comprising at least one radiation naïve patient and wherein the analysis comprises processing features of the one or more images, the features comprising radiomic features, wherein the radiomic features correlate to a structural integrity of one or more segments of the one or more vertebrae.

Provided herein is an apparatus for predicting risks of fracture in a patient. An apparatus may include a processor and a memory communicatively connected to the processor, the memory containing instructions configuring the processor to perform various functions. A processor may be configured to obtain image data of a patient. A processor may be configured to execute a machine learning model using image data as input, wherein the machine learning model is trained to identify one or more vertebrae of the image data, wherein the machine learning model is trained on a cohort of a population comprising at least one radiation naïve patient. A machine learning model may analyze one or more vertebrae to predict a future risk of vertebrae fracture of the one or more vertebrae for a patient. A machine learning model may be trained to process features of one or more images, wherein radiomic features correlate to a structural integrity of one or more segments of the one or more vertebrae.

Provided herein is a computer-implemented method for predicting risks of vertebrae fracture in a patient. A computer-implemented method may include obtaining image data of a patient. A computer-implemented method may include identifying, by a machine learning model, one or more vertebrae of image data, wherein the machine learning model is trained on a cohort of a population comprising at least one radiation naïve patient. A computer-implemented method may include analyzing, by a machine learning model, one or more vertebrae to predict a future risk of vertebrae fracture of the one or more vertebrae for a patient, wherein the analysis comprises processing features of one or more images, the features comprising radiomic features, wherein the machine learning model has a sensitivity of about 0.8 or greater.

The above and other preferred features, including various details of implementation and combination of elements, will now be more particularly described with reference to the accompanying drawings and pointed out in the claims. It will be understood that the particular methods and apparatuses are shown by way of illustration only and not as limitations. As will be understood by those skilled in the art, the principles and features explained herein may be employed in various and numerous embodiments.

### BRIEF DESCRIPTION OF THE DRAWINGS

The disclosed embodiments have advantages and features which will be more readily apparent from the detailed description, the appended claims, and the accompanying figures (or drawings). A brief introduction of the figures is below.

FIG. 1 illustrates a block diagram of a system for predicting risk of vertebrae fracture in a patient;

FIG. 2 illustrates a process of creating radiomic features;

FIG. 3 illustrates an example of implementation of methods and systems described herein with a cohort of patients;

FIG. 4 illustrates a computer-implemented method of predicting vertebrae fractures in patients;

FIG. 5 illustrates a computing system that may be used in systems and methods described herein;

FIG. 6 illustrates machine learning model that may be used in systems and methods described herein;

FIGS. 7A-D illustrates graphs showing results of models described herein using different variables; and

FIGS. 8A-B illustrate images of a vertebra before and after fracturing.

### DETAILED DESCRIPTION

Reference will now be made in detail to several embodiments, examples of which are illustrated in the accompanying figures. The Figures (Figs.) and the following description relate to preferred embodiments by way of illustration only. It is noted that wherever practicable similar or like reference numbers may be use in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

Spinal metastases are common in patients with metastatic cancer, approaching 70% across histologies. More patients with metastatic disease are living longer as systemic therapy, radiation, and surgical options improve. As a result of this, the sequelae of spinal metastases are becoming a greater problem for patient quality of life. A common consequence of spinal metastases is pathologic compression fracture. These fractures result in a significant increase in morbidity and mortality. Furthermore, they may progress to unstable fractures resulting in neurologic compromise, and require large reconstructive and stabilizing surgeries.

The ability to predict which tumor-infiltrated vertebrae will develop a fracture or undergo fracture progression is of significant clinical utility for all physicians who play a role in the care of these patients, including spine surgeons, oncologists, radiation-oncologists, primary care/internal medicine physicians, and emergency medicine practitioners. From a spine surgery perspective, consults for spinal metastases are frequent, especially amongst cancer hospitals. While the majority of patients do not require surgery at the time of consult, they often require close follow-up to assess worsening back pain, neurologic deficit, or new/progressive fracture that may require surgery. Better quantifying this risk would allow the surgeon to more optimally time patient follow up, reimaging, and intervention. From the radiation oncology standpoint, if deemed high risk for fracture, some patients would benefit from prophylactic intervention with vertebroplasty/kyphoplasty and/or percutaneous fixation before their indicated radiation.

Computer vision has shown promise in predicting vertebral insufficiency fractures. However, applications to predicting metastatic/tumor related vertebral fractures has been limited, and the existing studies have multiple limitations. For instance, computer vision models may be specific to a single cohort of patients, such as Stereotactic Body Radiation Therapy (SBRT) patients. While SBRT does carry one of the highest risks for subsequent fracture, many patients are at risk for fracture before radiation and these patients require equal attention and risk stratification. Second, computer vision models may utilize both Computed Tomography (CT) and Magnetic Resonance Imaging (MRI) radiomics, as well as both clinical and treatment data such as Eastern Cooperative Oncology Group (ECOG) scores and radiation dosing. However, MRIs are not always available or able to be obtained for some patients, and are expensive imaging modalities compared to CT. As such, MRI radiomic requirements make current models less generalizable, and theoretically more expensive, to all patients at risk for vertebral compression fracture (VCF). Moreover, those who are radiation-naïve will not have dosimetric data to be inputted into current models.

Aspects of the present disclosure may provide for predicting risks of fracture of one or more vertebrae using machine learning models. In some embodiments, methods and systems described herein may allow for prediction of vertebral body fracture (VBF) within a one year timeline. In some embodiments, predictions of risks of fractures may be made based on developing spinal metastases in patients. In some embodiments, machine learning models describe herein may be trained to use exclusively CT image data, eliminating the need for incorporation of MRI or other imaging data. Machine learning models described herein may be trained on a cohort of patients including at least one radiation-naïve patient, which may overcome limitations of current models in predicting spinal fractures by eliminating restriction to use of dosimetric data while achieving similar or better prediction accuracy scores.

### OVERVIEW

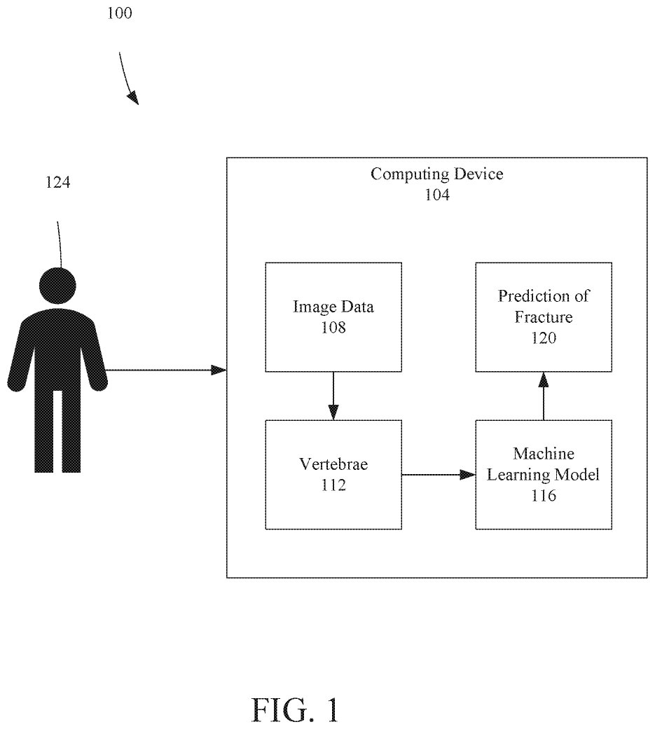

Referring now to FIG. 1, a system 100 for predicting vertebrae fractures is presented. System 100 may include computing device 104. Computing device 104 may include a processor and a memory communicatively coupled to the processor. Computing device 104 may be, but is not limited to, a server, desktop, laptop, or other computing device. In some embodiments, computing device 104 may operate on a network. A network may include, but is not limited to, a Wi-Fi, Bluetooth, and/or cellular network.

In some embodiments, computing device 104 may be configured to obtain image data 108 of patient 124. “Image data” as used in this disclosure refers to any information of an object and/or individual pertaining to the electromagnetic spectrum. Image data 108 may include, but is not limited to, pixel values, red green blue (RGB) values, cyan magenta yellow black (CMYK) values, brightness, contrast, sharpness, exposure, voxel data, depth maps, point clouds, or other forms of data. For instance and without limitation, image data 108 may include images of one or more parts of patient 124. Parts may include, but are not limited to, torsos, backs, ribs, skulls, feet, hands, or other parts of the human body. Image data 108 may include, but is not limited to, X-ray images, computed tomography (CT) images, MRI images, or other forms of imagery. Image data 108 may be generated from patient 124 through one or more imaging systems. Imaging systems may include, but are not limited to, X-ray machines, CT imaging machines, cameras, or other imaging systems. In some embodiments, image data 108 may include visible light, X-ray, and/or CT photographs of a portion of patient 124. Image data 108 may pertain to one or more vertebrae 112 of patient 124. For instance, image data 108 may capture one or more vertebral structures within patient 124.

Image data 108 may include one or more voxels. A “voxel” as used in this disclosure refers to a volumetric pixel. A voxel may be a three-dimensional (3D) pixel, in some embodiments. For instance, a voxel may have a width, length, and height value, whereas a pixel may have width and length value. As a non-limiting example, image data 108 may include a slice of a CT scan with a thickness of about 1 mm, where a pixel may have a width of about 0.5 mm and a length of about 0.5 mm, and a voxel may have a width of about 0.5 mm, a length of about 0.5 mm, and a height of about 1.0 mm. Computing device 104 may be configured to input a plurality of voxels of image data 108 and determine vertebrae 112 based on the plurality of voxels. In some embodiments, computing device 104 may be configured to identify one or more vertebrae 112 within image data 108. “Vertebrae” as used in this disclosure refers to an interconnected bone that forms the vertebral column. Vertebrae may include any structure of the spine, such as, but not limited to, vertebral body, vertebral arch, spinous process, transverse processes, facet joints, intervertebral discs, or other portions of the spine and/or spinal column.

In some embodiments, computing device 104 may utilize machine learning model 116 to identify one or more vertebrae 112 within image data 108. Machine learning model 116 may be but is not limited to, a random forest classifier, logistic regression classifier, support vector machine, Naïve Bayes model, K-nearest neighbors (KNN) model, or other type of machine learning model. Machine learning model 116 may be trained with training data correlating images to identified and/or annotated vertebrae. Training data may be received via user input, external computing devices, and/or previous iterations of processing. In some embodiments, vertebrae 112 may be identified and/or annotated manually. Machine learning model 116 may input image data 108 and output identified vertebrae 112. Identified vertebrae 112 may include segmented pixels of image data 108 representative of one or more portions of vertebrae 112 of patient 124. “Segmentation” as used in this disclosure refers to the processing of dividing an object into one or more parts. For instance, vertebrae 112 may be segmented into cervical, thoracic, lumbar, sacral, and/or coccygeal regions. In some embodiments, each individual structure of vertebrae 112 may be segmented. In other embodiments, groups of two or more structures of vertebrae 112 may be segmented. Segmentation or other identification of vertebrae 112 may be provided to a medical professional or other user through a display, which may aid the medical professional or other user in examining vertebrae 112 of patient 124. In some embodiments, vertebrae 112 may include vertebral bodies, vertebral arches, vertebrae processes, vertebrae foramen and/or canal, intervertebral discs, specialized vertebra, or other forms of vertebrae. In some embodiments, computing device 104 may be configured to identify each vertebrae 112 within image data 108 and may annotate image data 108 to identify each vertebrae 112. Computing device 104 may be configured to identify one or more vertebral levels. A “vertebral level” as used in this disclosure refers to a segment of the spine that corresponds to a particular vertebra. For instance and without limitation, vertebral levels may include C1-C7, T1-T12, L1-L5, S1-S5, or other vertebrae levels.

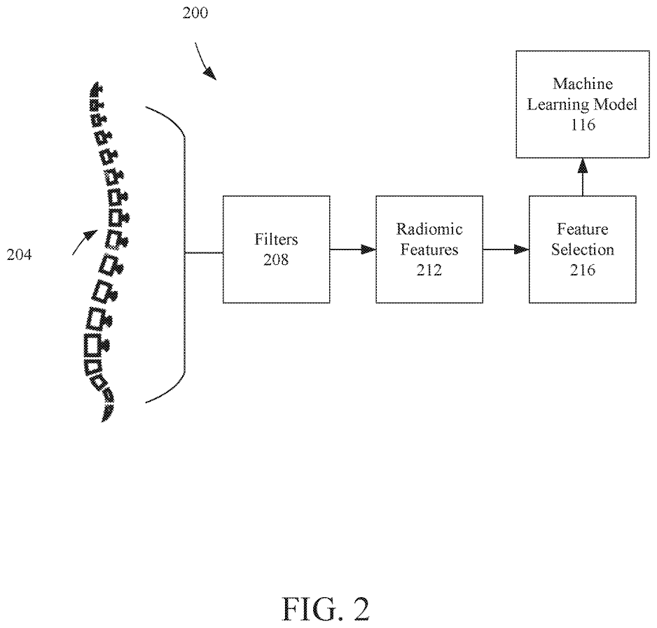

Still referring to FIG. 1, machine learning model 116 may be configured to extract one or more features from identified vertebrae 112 within image data 108. Features may include, but are not limited to, radiomic features, clinical features, or other types of features, which may be as described below with reference to FIG. 2. In some embodiments, machine learning model 116 may process one or more features of image data 108 and/or vertebrae 112 to generate a prediction of fracture 120. A “prediction of fracture” as used in this disclosure refers to a probability of failing structural integrity in a bone. For instance, machine learning model 116 may output prediction of fracture 120 which may include a probability of one or more identified vertebrae 112 fracturing within a timeline. A timeline may include a period of time of about, but not limited to, days, weeks, months, a year, or greater than a year. Probabilities may include any percentage value between about 0% and about 100%, without limitation. In some embodiments, prediction of fracture 120 may be specific to a location of vertebrae 112. A location may include any vertebrae within the C1-C7, T1-T12, L1-L5, S1-S5, or other vertebrae levels. For instance and without limitation, a vertebrae within T1 through L5 levels may be identified as having a prediction of fracture 120. In some embodiments, prediction of fracture 120 may include a new fracture within one or more identified vertebrae 112. In some embodiments, prediction of fracture 120 may include a progression of a pre-existing fracture identified in vertebrae 112. For instance, machine learning model 116 may be trained to identify one or more pre-existing fractures of vertebrae 112. Machine learning model 116 may input a presence and/or severity of a pre-existing fracture in generating prediction of fracture 120. A presence of a fracture may include an identification of one or more pre-existing fractures within patient 124. In some embodiments, a classifier separate from machine learning model 116 may be utilized to input image date 108 and outputs presences or absences of pre-existing vertebral fractures. A classifier may be trained with training data correlating image data 108 to presences or absences of vertebral fractures. A “pre-existing fracture” as used in this disclosure refers to a vertebral fracture that is present or was present at a current time. For instance, patient 124 may have a current vertebral fracture and/or a vertebral fracture that may have healed in the past. Machine learning model 116 may be trained to identify a healed fracture, in some embodiments. For instance training data may be provided to machine learning model 116 correlating image data to healed vertebral fractures. A “healed vertebral fracture” as used in this disclosure refers to a previous vertebra fracture that has been mended by the human body. A healed vertebral fracture may increase a bone density around a fracture site, in some embodiments. Machine learning model 116 may be trained to identify healed fractures and/or pre-existing fractures within patient 124 and incorporate the healed fractures into determining prediction of fracture 120. For instance, a first vertebrae 112 may be identified as having a pre-existing fracture while a second vertebrae positioned adjacent the first vertebrae may have an increased prediction of fracture 120 due to the pre-existing condition of the first vertebrae, relative to if the first vertebrae was absent a pre-existing fracture.

In some embodiments, prediction of fracture 120 may include a vertebral body fracture (VBF), such as, but not limited to, a compression fracture, burst fracture, wedge fracture, chance fracture, or other type of fracture. A “compression fracture” as used in this disclosure refers to type of spinal fracture in which part of the spine collapses. A “burst fracture” as used in this disclosure is a type of spinal fracture in which part of the spine is fractured into multiple pieces. A “wedge fracture” as used in this disclosure is a type of spinal fracture when part of the spine becomes wedge-shaped. For instance, a front part of a vertebra may collapse or compress, which may cause the vertebra to become wedge-shaped. A “chance fracture” as used in this disclosure is a type of spinal fracture in which part of the spine is flexed or flexed while being pulled apart. Machine learning model 116 may be trained to generate prediction of fracture 120 including a category of fracture. A “category of fracture” as used in this disclosure refers to a classification of a loss of structural integrity in a bone. A category of fracture may include, but is not limited to, a compression fracture, burst fracture, wedge fracture, chance fracture, or other type of fracture. In some embodiments, a category of fracture may be a severity level. A “severity level” as used in this disclosure refers to a relative stage of a fracture. A severity level may include, but is not limited to, mild, moderate, severe, extremely severe, or other levels. A mild severity level may include a fracture having a width of about less than 2 mm, such as but not limited to a hairline fracture or wedge compression fracture. A moderate severity level may include a fracture having a width of greater than about 2 mm, such as but not limited to a compression fracture, greenstick fracture, or other type of fracture. A severe severity level may include a fracture having a width of greater than about 5 mm. An extremely severe severity level may include a fracture having a width of greater than about 10 mm. In some embodiments, severity levels may be based on a total height loss of one or more vertebra. A “height loss” as used in this disclosure refers to a reduction in vertical size of a vertebra. For instance a mild severity level may have a height loss of about 0% to about 20%. A moderate severity level may have a height loss of about 20% to about 40%. A severe severity level may have a height loss of about 40% to about 70%. An extremely severe severity level may have a height loss of more than about 70%.

In some embodiments, machine learning model 116 may identify one or more tumors within image data 108. A “tumor” as used in this disclosure refers to an abnormal growth in the human body. Tumors may include vertebral tumors, such as, but not limited to, osteoid osteoma, osteoblastoma, hemangioma, giant cell tumor (GCT), aneurysmal bone cyst (ABC), chordoma, chondrosarcoma, Ewing sarcoma, osteosarcoma, or other forms of tumors. Tumors may have infiltrated one or more vertebrae 112. For instance, tumors may grow inside of and/or adjacent to any vertebrae described throughout this disclosure, without limitation. Machine learning model 116 may be trained to identify a type of tumor determined to be present within image data 108. For instance, machine learning model 116 may be trained with training data correlating images of tumor-infiltrated vertebrae to classifications of tumors. Training data may be received via user input, external computing devices, and/or previous iterations of processing. Machine learning model 116 may be trained to generate prediction of fracture 120 based on tumor data identified within image data 108. Machine learning model 116 may be trained to identify tumor data with image data 108. “Tumor data” as used in this disclosure refers to any information pertaining to an abnormal growth within the human body. For instance, tumor data may include, but is not limited to, size, shape, density, location, or other data relating to one or more tumors within a patient. Machine learning model 116 may incorporate tumor data extracted from image data 108 in generating prediction of fracture 120. In some embodiments, prediction of fracture 120 may be generated based on a prediction of tumor growth within patient 124. For instance, machine learning model 116 may determine a projected growth of one or more tumors within the human body. In some embodiments, machine learning model 116 may determine a projected size and/or shape of a tumor over a period of time, which may displace or otherwise impact one or more adjacent vertebrae 112. Machine learning model 116 may determine tumor types, tumor sizes, tumor shapes, projected growth, projected sizes, and/or projected shapes of a tumor identified in image data 108 and may utilize the prior described data in generating prediction of fracture 120. For instance, an identified aggressive tumor may correlate to prediction of fracture 120 having a higher probability relative to image data 108 absent any tumor.

In some embodiments, machine learning model 116 may be trained to annotated one or more identified vertebrae 112 with a prediction of fracture 120 greater than about 0%. Annotation of vertebrae 112 with predictions of fractures 120 may be displayed through a display device in communication with computing device 104. A display device may include, but is not limited to, a monitor, television screen, smartphone screen, laptop screen, tablet screen, or other type of display. For instance, machine learning model 116 may highlight and/or annotate one or more pixels and/or voxels of image data 108 that may be representative of vertebrae 112. In some embodiments, machine learning model 116 may utilize a color coding system in annotating image data 108. A “color coding system” as used in this disclosure refers to a process of indicating data of two or more objects based on different pixel color values. For instance, a color coding system may include a first color representative of a healthy vertebrae 112, a second color representative of a tumor infiltrated vertebrae 112, and/or a third color representative of a fracture vertebrae 112. In some embodiments, a color coding system may include four or more colors.

In some embodiments, image data 108 may be exclusively CT imagery. For instance, image data 108 may be entirely CT images, absent of X-ray or other imagery. Machine learning model 116 may be trained with training data correlating CT images to vertebrae and/or predicted risks of future fractures. Training data may be received via user input, external computing devices, and/or previous iterations of processing. One or more features utilized by machine learning model 116 in generating prediction of fracture 120 and/or identification of vertebrae 112 may be extracted solely from CT images, in some embodiments. For instance, machine learning model 116 may be trained to input image data 108 obtained from one or more CT images and output prediction of fracture 120. In some embodiments, machine learning model 116 may be trained on training data having between about 50% CT imagery to about 60% CT imagery, about 60% CT imagery to about 70% CT imagery, about 70% CT imagery to about 80% CT imagery, about 80% CT imagery to about 90% CT imagery, or about 90% CT imagery to about 100% CT imagery. In some embodiments, machine learning model 116 may be trained on image data 108 having a resolution of about or less than about 1920×1080.

Computing device 104 may be in communication with a third-party network. A “third-party network” as used in this disclosure refers to one or more computing systems external to computing device 104. For instance, computing device 104 may be in communication with a local area network (LAN), Wi-Fi, or other network. Computing device 104 may be in communication with a hospital network. For instance, computing device 104 may obtain image data 108 of one or more patients 124 through a computing device of a hospital network. Computing device 104 may identify vertebrae 112 and/or generate prediction of fracture 120 and may communicate identified vertebrae 112 and/or predictions of fracture 120 to one or more computing devices in a hospital network.

### Radiomic Features and Feature Selection

Referring now to FIG. 2, a process 200 of generating one or more radiomic features 212 is presented. Process 200 may include obtaining vertebral image 204. Vertebral image 204 may be a CT image, MRI image, X-ray image, or other type of image. In some embodiments, vertebral image 204 may be an entire spinal column of a patient. In some embodiments, vertebral image 204 may be a portion of one or more vertebral structures of a patient. Vertebral image 204 may have an orientation. For instance, relative to a capture point, one or more vertebral structures within vertebral image 204 may be rotated at any angle along a horizontal axis of vertebral image 204. For instance, one or more vertebral structures within vertebral image 204 may be rotated by about, but not limited to, about 10 degrees to about 20 degrees, about 20 degrees to about 30 degrees, about 30 degrees to about 40 degrees, about 40 degrees to about 50 degrees, about 50 degrees to about 60 degrees, about 60 degrees to about 70 degrees, about 70 degrees to about 80 degrees, about 80 degrees to about 90 degrees, about 90 degrees to about 100 degrees, about 100 degrees to about 180 degrees, or about 180 degrees to about 360 degrees. In some embodiments, one or more vertebral structures within vertebral image 204 may be rotated along a vertical axis of vertebral image 204. An angle of rotation along a vertical axis of vertebral image 204 may be about, but not limited to, about 10 degrees to about 20 degrees, about 20 degrees to about 30 degrees, about 30 degrees to about 40 degrees, about 40 degrees to about 50 degrees, about 50 degrees to about 60 degrees, about 60 degrees to about 70 degrees, about 70 degrees to about 80 degrees, about 80 degrees to about 90 degrees, about 90 degrees to about 100 degrees, about 100 degrees to about 180 degrees, or about 180 degrees to about 360 degrees.

In some embodiments, vertebral image 204 may have a resolution. A “resolution” as used in this disclosure refers to an images pixel width by pixel height. For instance, and without limitation, vertebral image 204 may have a resolution of about 1280×720, 1920×1080, 2560×1440, or greater than 2560×1440. Vertebral image 204 may have a contrast. A “contrast” as used in this disclosure refers to a difference in brightness and/or color in an image. Contrasts may include luminance contrasts and/or color contrasts. In some embodiments, vertebral image 204 may have a contrast ratio, such as, but not limited to, about 100:1 to about 500:1, about 500:1 to about 5000:1, or greater than about 5000:1. In some embodiments, vertebral image 204 may have a sharpness. A “sharpness” as used in this disclosure refers to a clarify of detail within an image. Sharpness may include, but are not limited to, blurry, soft, crisp, or other levels of sharpness. For instance and without limitation, in a CT image, pixel values may be spread over a wide range of about 50 to about 250 Hounsfield Units (HU), which may make it difficult to distinguish between soft tissue and tumors. In some embodiments, vertebral image 204 may be in gray-scale. “Gray-scale” refers to images that are only one or more of black, white, and gray. For instance, vertebral image 204 may be in gray-scale with dense tissues, such as but not limited to bones, appearing white, soft tissues, such as but not limited to muscles and organs, appearing gray, and air-filled spaces, such as but not limited to lungs or surroundings, appearing black.

Process 200 may include applying one or more filters 208 to vertebral image 204. In some embodiments, filters 208 applied to vertebral image 204 may modify one or more characteristics of vertebral image 204. For instance, and without limitation, filters 208 may increase a contrast, brightness, sharpness, resolution, or other characteristic of vertebral image 204. Filters 208 may remove noise from vertebral image 204. “Noise” as used in this disclosure refers to disruptive signals in an image. For instance, noise may cause an image to be blurry or otherwise distorted. A “filter” as used in this disclosure refers to a component that removes certain data, such as but not limited to noise. For instance, filters 208 may include one or more of, but not limited to, wavelet, square, square root, logarithm, exponential, Laplacian of Gaussian, gradient, local binary pattern 3D, and/or other filters.

A wavelet filter may be a filter that decomposes a signal or image into one or more frequency bands using one or more wavelet transforms, filters out noise, and reconstructs a cleaned signal or image. Wavelet transforms may include, but are not limited to, Discrete Wavelet Transform (DWT), Stationary Wavelet Transform (SWT) or other forms of transforms. A wavelet filter may separate an image into low-frequency components and high-frequency components. Low-frequency components may be overall structure of regions of interest in an image, while high-frequency components may be edges, textures, and/or noise.

A square filter may be a filter that applies a fixed square-shaped kernel or window to an image or signal to process data. A square signal may include, but is not limited to, a mean filter, median filter, Gaussian filter, edge detection filter, or other filter. A square filter may be a matrix of coefficients that may be applied to an image. A matrix of coefficients may be an N×N matrix, with N being any number. As a non-limiting example, a matrix of coefficients may be 3×3, 5×5, or other matrices. A matrix of coefficients may be used to manipulate one or more pixel values of vertebral image 204. For instance, a square filter may utilize convolution which may involve moving across an image pixel by pixel and multiplying values of the square filter with pixel values of the image, resulting in summed pixel values that produce a new pixel value for a central pixel of the filter. Convolution using a square filter may be repeated for every pixel in an image.

A square root filter may be a non-linear filter that may apply a square root to a pixel value of a pixel. For instance, a square root filter may apply a square root to a pixel intensity value of one or more pixels, which may result in a darker pixel. A square root filter may be utilized to compress a dynamic range of an image, which may reduce high bright spots in an image. For instance, a square root filter may allow for emphasizing of details within one or more brighter regions of an image without losing details in one or more darker regions of the image. A square root filter may adjust an overall contrast of an image.

A logarithmic filter may perform a logarithmic transformation of one or more pixel values within an image. For instance, each pixel of an image may go through a logarithmic transformation, which may emphasize lower intensity values and may compress higher intensity values. A logarithmic filter may enhance low-contrast images, compress dynamic range, and/or highlight darker areas within an image.

An exponential filter may apply an exponential transformation to a pixel value of an image. For instance, each pixel value within an image may go through an exponential transformation. An exponential transformation of a pixel value may enhance brighter regions of an image while compressing intensity values of darker regions within an image.

A Laplacian of Gaussian (LoG) filter may be a two-step process that may combine a Laplacian operation and a Gaussian filter. An LoG filter may apply a Gaussian filter first, which may smooth out an image similarly to applying a low-pass filter to the image. An LoG filter may apply a Laplacian operator after applying a Gaussian filter, which may be a second-order derivative filter that detects regions of rapid intensity change, such as but not limited to edges, in an image.

A gradient filter may apply a horizontal and/or vertical gradient to one or more pixels of an image. For instance, a horizontal gradient may represent a change of values in an X-direction, while a vertical gradient may represent a change in values in a Y-direction. A gradient filter may apply a first derivative of one or more pixel values in both horizontal and vertical directions. Gradient filters may include, but are not limited to, Sobel filters, Prewitt filters, Scharr filters, or other gradient filters. Gradient filters may assist in sharpening bright-to-dark edges and dark-to-bright edges. In some embodiments, gradient filters may assist in detecting corners, lines, and/or boundaries of one or more objects within an image. Gradient filters may provide orientations or directions of edges, such as by giving a direction of a gradient vector.

A local binary pattern 3D filter may be a texture descriptor. For instance, a local binary pattern 3D filter may compare one or more voxels of an image. A binary pattern may be generated for each voxel in an image by comparing it with one or more neighboring voxels. Each comparison may generate a bit, such as 0 or 1. A resulting binary pattern for each voxel may be converted into a decimal value, which may represent a local texture or pattern. A histogram of decimal values over an image and/or volume may be calculated, which may be used as a feature vector to characterize a texture of the volume and/or image.

With continued reference to FIG. 2, in some embodiments, a single filter may be used on vertebral image 204. In other embodiments, two or more filters 208 may be used on vertebral image 204. Application of filters 208 to vertebral image 204 may modify image data of vertebral image 204. For instance, filters 208 may remove unwanted noise above or below a threshold frequency level, which may improve image quality of vertebral image 204. A threshold frequency level may be about 0.1 to about 0.3 of the Nyquist frequency of vertebral image 204. A “Nyquist frequency” as used in this disclosure refers to half of a sampling rate of a signal. A Nyquist frequency may represent a highest frequency that may be captured without aliasing. “Aliasing” as used in this disclosure refers to distortion that occurs to an image or signal that occurs when the image or signal is sampled at an insufficient rate.

Process 200 may include extracting radiomic features 212 from vertebral image 204. A “radiomic feature” as used in this disclosure refers to data relating to pixel and/or voxel values associated with medical images. For instance and without limitation, radiomic features may include shaped-based features. “Shape-based features” as used in this disclosure refer to parameters relating to a geometry of an object. Shape-based features may include, but are not limited to, a volume of a region of interest, a surface area of a region of interest, a sphericity of a region of interest, a compactness of a region of interest, an elongation of a region of interest. Radiomic features 212 may include intensity-based features. “Intensity-based features” as used in this disclosure refers to a magnitude of one or more pixel values of an image. Intensity-based features may include, but are not limited to, mean intensity within a region of interest, standard deviation of intensity within a region of interest, skewness, kurtosis, or other features. Radiomic features 212 may include texture-based features. “Texture-based features” as used in this disclosure refer to surface parameters of an object. Texture-based features may include, but are not limited to, gray-level co-occurrence matrix (GLCM), contrast, correlation, energy, homogeneity, gray-level run length matrix (GLRLM), local binary pattern (LBP), gray-level size zone matrix (GLSZM) or other features. Radiomic features 212 may include statistical features. “Statistical features” as used in this disclosure refer to spatial relationships between objects within an image. Statistical features may include, but are not limited to, entropy, mean, variance, skewness, kurtosis, or other features. Radiomic features 212 may include wavelet-based features. “Wavelet-based features” as used in this disclosure refer to parameters relating to one or more frequency bands of an image. Wavelet-based features may include, but are not limited to, wavelet transform, Haar wavelet, or other features. Radiomic features 212 may include higher-order features. “Higher-order features” as used in this disclosure refer to complex parameters of an image. Higher-order features may include, but are not limited to, fractal dimension, run-length matrix, or other features. In some embodiments, radiomic features 212 may be extracted directly from vertebral image 204. In other embodiments, radiomic features 212 may be extracted through a filtered version of vertebral image 204 through an application of one or more filters 208.

Radiomic features 212 may include a single class of features, such as, but not limited to, shape-based, intensity-based, texture-based, or other feature classes. In some embodiments, radiomic features 212 may include a combination of two or more of shape-based, intensity-based, texture-based, wavelet-based, statistical, or any other form of feature described herein. Radiomic features 212 may include a plurality of features. For instance, radiomic features 212 may include between about 1 feature to about 2,050 features or greater than about 2,050 features. In some embodiments, radiomic features 212 may have a higher ratio of one or more feature classes compared to one or more other feature classes. For instance, there may be a ratio of about 1:100 to about 100:1 of any parameter described herein with any other parameter described herein. As a non-limiting example, a set of radiomic features 212 may include about 14 shaped-based features, 18 intensity-based features, and 75 texture-based features.

In some embodiments, radiometric features 212 may be selected through feature selection 216. “Feature selection” as used in this disclosure refers to a process of retaining or removing one or more parameters. Feature selection 216 may include, but is not limited to, recursive feature elimination (RFE). “Recursive feature elimination” as used in this disclosure refers to a process of selecting feature that recursively removes a least important feature from a dataset. In some embodiments, feature selection 216 may include a filter method, wrapper method, embedded method, dimensionality reduction, genetic algorithms, unsupervised feature selection, or other forms of feature selection. Feature selection 216 may include processing one or more radiomic features 212 to identify a top number of radiomic features 212 having a relative highest importance. A “relative highest importance” as used in this discourse refers to a weight of a parameter that is greater than one or more weights of one or more other parameters. For instance, a texture-based parameter may have a weight of about 0.4, while a shaped-based and intensity-based parameter may each have a weight of about 0.3, indicating the texture-based parameter has a relative highest importance. In some embodiments, feature selection 216 may include identifying highly correlated features based on a correlation coefficient. A “correlation coefficient” as used in this disclosure refers to a value that is indicative of a correlation between two or more variables. A correlation coefficient may be any value between about 0 and about 1, with 1 representing a highest correlation. In some embodiments, feature selection 216 may filter out any radiomic features 212 beneath a threshold correlation coefficient, such as, but not limited to, about 0.9 to about 0.8, about 0.8 to about 0.7, about 0.7 to about 0.6, or less than about 0.6.

Feature selection 216 may select a top 5, top 10, top 20, or greater than top 20 radiomic features 212 out of a plurality of radiomic features 212. Feature selection 216 may select one or more radiomic features 212 that may correlate to vertebrae data. “Vertebrae data” as used in this disclosure refers to any information pertaining to the spinal column. Vertebrae data may include, but is not limited to, vertebrae width, vertebrae size, vertebrae shape, vertebrae strength, vertebrae location, or other forms of vertebrae data. For instance, texture-based and/or shape-based features may be representative of trabecular bone, osteoporotic bone, and/or tumors.

Still referring to FIG. 2, machine learning model 116 may be trained to input one or more features selected by feature selection 216. For instance, machine learning model 116 may input a top 5, top 10, top 20, or greater than a top 20 amount of radiomic features 212 selected by feature selection 216. Machine learning model 116 may input any class of feature described herein, such as, but not limited to, texture-based features, shape-based features, intensity-based features, or other feature types. Machine learning model 116 may identify one or more radiomic features 212 that may correspond to vertebra data. For instance, machine learning model 116 may identify that one or more texture-based and/or shaped-based features correspond to structural integrity of a portion of a vertebra. A “structural integrity” as used in this disclosure refers to an ability of a material to withstand a mechanical load. For instance, a vertebral structure may be able to withstand a mechanical load applied by its own weight and may stay intact, or may not be able to withstand a mechanical load applied by its own weight and may fracture. Machine learning model 116 may identify a structural integrity of one or more portions of a vertebrae based on one or more radiomic features 112. Machine learning model 116 may be trained to consider one or more forces that may be applied to one or more vertebrae, and/or radiomic features 112 that may correspond to one or more forces. Force data may be part of training data that machine learning model 116 may be trained on. For instance, training data may correlate various structures within one or more vertebral images 204 to one or more forces, amounts of forces, predicted changes in experience of forces, or other parameters. Forces may include, but are not limited to, tensile strength, stress, compressive force, shear force, torsional force, bending force, or other forces. Machine learning model 116 may be trained to identify portions of a vertebral structure that may be experiencing or may be predicted to experience one or more forces. In some embodiments, machine learning model 116 may be trained to identify an increase, decrease, or unchanging amount of force applied to one or more parts of a vertebra. In some embodiments, machine learning model 116 may correlate one or more forces to increases or decreases in predicted fractures of a vertebra. For instance, a higher calculated tensile force may be correlated to a higher probability of a future fracture within a vertebral body, while a torsional force may have a lesser increase in probability of a future fracture within a vertebral body compared to an increase in tensile strength.

In some embodiments, radiomic features 212 may include one or more of voxel volume, shape elongation, shape flatness, maximum 2D diameter row, shape sphericity, first order 10^{th} percentile, mean absolute deviation, first order mean, and first order median. Machine learning model 116 may be trained with training data correlating any one or all of voxel volume, shape elongation, shape flatness, maximum 2D diameter row, shape sphericity, first order 10^{th} percentile, mean absolute deviation, first order mean, and first order median to future risks of vertebrae fracture. Training data may be received via user input, external computing devices, and/or previous iterations of processing. “Voxel volume” as used in this disclosure refers to a volumetric area of a one or more voxels. One or more voxels may be representative of, but not limited to, a tumor, vertebra, organ, or other part of the human body. Voxel volume may be represented as cubic millimeters, cubic inches, or other measurements. Voxel volume may be calculated for tumors and/or vertebrae within an image. In some embodiments, voxel volume may be used by machine learning model 116 to generate predictions of future risks of fracture of one or more vertebra. For instance, machine learning model 116 may correlate a higher voxel volume of a tumor with an increase in a risk of future vertebrae fracture with one or more vertebrae adjacent of the tumor. Machine learning model 116 may correlate voxel volumes of vertebrae with increases or decreases in future risks of vertebrae fracture. For instance, a vertebrae having a smaller voxel volume compared to one or more other vertebrae may be correlated to a higher probability of future risks of vertebrae fracture than one or more vertebrae having higher voxel volumes. A vertebrae having a larger voxel volume compared to one or more other vertebrae may have a smaller probability of future risks of vertebrae fracture than one or more vertebrae having smaller voxel volumes. Voxel volumes may correlate to structural integrity of a vertebrae. For instance and without limitation, a higher voxel volume may correlate to a higher structural integrity of a vertebrae compared to a vertebrae with a lesser voxel volume. In some embodiments, machine learning model 116 may identify correlations between voxel volumes of two or more adjacent vertebrae. For instance, a greater voxel volume of a first vertebrae compared to a second adjacent vertebrae having a smaller voxel volume may correlate to the second adjacent vertebrae having an increase in future risks of fracture. A first vertebrae having a higher voxel volume than a second vertebrae may correspond to the first vertebrae applying a strong force on the spinal column and/or the second vertebrae, which may correlate to the second vertebrae having an increased probability of future vertebrae fracture. A vertebrae having a smaller voxel volume compared to one or more adjacent vertebrae may correlate to an increase in future risk of vertebral fracture on the vertebrae or surrounding vertebral structures, as the vertebrae may have a voxel volume that is unable to structurally support surrounding vertebral structures. In some embodiments, a high voxel volume of a tumor and a low voxel volume of an adjacent vertebrae may be correlated to a higher probability of future risks of vertebrae fracture. For instance, a large tumor may apply a force on a smaller vertebrae comparted to one or more other vertebrae, which may compromise a structural integrity of the smaller vertebra. Machine learning model 116 may be trained to calculate any correlation between voxel volume and future risk of vertebra fracture described herein.

“Shape elongation” as used in this disclosure refers to a geometry of a 3D or 2D object. 3D or 2D objects may include but are not limited to organs, vertebrae, and/or tumors. Shape elongation of a tumor and/or vertebra may correlate to increases or decrease of future risks of vertebrae fracture. For instance, shape elongation of one or more vertebrae may indicate a misalignment or abnormal geometry of the one or more vertebrae. For instance, a first vertebrae may be aligned properly, while a second vertebrae along the spinal column and/or adjacent to the first vertebrae may be misaligned. Misalignment may include a vertical and/or horizontal displacement of a vertebrae relative to a longitudinal and/or lateral axis of a patients spinal column. For instance, a vertebrae may be displaced along a longitudinal axis relative to a central axis of a spinal column by about 1 mm to about 10 mm or greater. A vertebrae may be displaced along a lateral axis relative to a central axis of a spinal column by about 1 mm to about 10 mm or greater. A misalignment may include a rotation of a vertebrae. For instance, a vertebrae may be rotated relative to a central axis of a patients spinal column by about, but not limited to, about 0 degrees to about 5 degrees, about 5 degrees to about 10 degrees, about 10 degrees to about 15 degrees, about 15 degrees to about 20 degrees, about 20 degrees to about 25 degrees, about 25 degrees to about 30 degrees, about 30 degrees to about 35 degrees, about 35 degrees to about 40 degrees, about 40 degrees to about 45 degrees, or greater than about 45 degrees. For instance and without limitation, a vertebrae may be rotated by about 45 degrees to about 180 degrees or greater than about 180 degrees. In some embodiments, two or more vertebrae may be identified as being misaligned. An abnormal geometry of a vertebrae may include one or more concavities, dents, deformed edges, heights, or other metrics of a vertebrae. For instance, a vertebrae may be abnormally short in height relative to one or more adjacent vertebrae, which may correlate to an increase in future risk of vertebral fracture. Machine learning model 116 may be trained to predict future risks of vertebrae fracture based on shape elongation of one or more of a tumor and vertebrae.

“Shape flatness” as used in this disclosure refers to how thin or disk-like a 3D object is. Machine learning model 116 may be trained to correlate shape flatness of a vertebrae and/or tumor to future risks of vertebrae fracture. For instance, a tumor and/or vertebrae may have a flatness that may correlate to increases or decreases in future risks of vertebral fracture. Flatness may be scored with a flatness value of between about 1 to about 0, with 1 being spherical or cub-like and 0 being flat. A vertebral structure that has a high flatness may have a weaker structural integrity, which may lead to an increase in future risks of vertebral fracture. A high shape flatness may increase a chance of a vertical fracture of a vertebrae.

“Maximum 2D diameter row” as used in this disclosure refers to a largest straight-line distance between any two points within a horizontal plane. For instance, a maximum 2D diameter row may be a distance between any two points of a 2D axial slice of a 3D region, such as of a tumor, vertebrae, and/or organ. A maximum 2D diameter row may be a distance between two points along a spinal column, a distance between a tumor and a vertebrae, or other distances. In some embodiments, a decreased maximum 2D diameter row relative to one or more other maximum 2D diameter rows may indicate anterior collapse and/or loss of vertebral body width, which may increase changes of wedge fractures in one or more vertebra. Anterior collapse may include a compression or collapse of a front portion of a vertebral body. Asymmetry in maximum 2D diameter row may indicate an irregular deformation, which may increase a probability of future risk of vertebral fracture. For instance, asymmetry may include a difference in width within a single axial slice of a vertebrae. As a non-limiting example, a normal vertebrae may have a maximum 2D diameter row on a left side of about 45 mm and on a right side of about 45 mm, while an asymmetric vertebrae may have a maximum 2D diameter row on a left side of about 40 mm and a maximum 2D diameter row on a right side of about 30 mm. Machine learning model 116 may be trained to correlate maximum 2D diameter row to future risks of vertebrae fracture.

“Shape sphericity” as used in this disclosure refers to how sphere-like a structure is. Shape sphericity may be calculated using a sphericity value of about 0 to about 1, with 1 being a perfect sphere and 0 being a highly elongated shape. In some embodiments, a moderate or high sphericity value may correlate to a healthy vertebral structure, while a flattened or irregular vertebral structure may have a low sphericity value. Malignant tumors may have a low sphericity value while benign tumors may have a higher sphericity. A higher sphericity value of one or more vertebrae may correlate to a decrease in future risks of vertebral fracture. A lower sphericity value of one or more vertebrae may correlate to an increase in future risks of vertebral fracture. A lower sphericity value of one or more tumors may correlate to a higher risks of future vertebral fracture. A higher sphericity value of one or more tumors may correlate to lower risks of future vertebral fracture.

“First order 10^{th} percentile” as used in this disclosure refers to a quantification of a distribution of pixel and/or voxel intensities in an image. A lower first order 10^{th} percentile value may be indicative of low-density structures within an image, while a higher first order 10^{th} percentile value may be indicative of higher-density structures within an image. Lower first order 10^{th} percentile values may correspond to bone loss and/or soft tissue development around a vertebral structure.

“Mean absolute deviation” as used in this disclosure refers to an averaging of absolute differences between two or more data points. Mean absolute deviation may be calculated for two or more pixel and/or voxel intensity values. Mean absolute deviation may correlate to how spread out voxel intensities are in an image. A lower mean absolute deviation value may indicate a more uniform texture or structure, while a higher mean absolute deviation value may indicate a greater variability in a texture or structure, such as within vertebral bodies, tumors, or other parts of the human body.

“First order mean” as used in this disclosure refers to an average intensity of a value of an image or region of interest. A first order mean may be calculated for pixel and/or voxel values. For instance, a first order mean may be calculated for pixel and/or voxel intensity values. A higher first order mean value may be indicative of denser tissues, such as bone, while a lower first order mean value may be indicative of less dense tissue, such as air spaces, tumors, or other low-density areas. For instance, fractured vertebrae may have a lower first order mean value than unfractured vertebrae. Tumors may cause a reduction in first order mean values due to the less dense tissue compared to surrounding vertebral structures.

“First order median” as used in this disclosure refers to a middle value of a dataset. For instance, a first order median may be a middle value of a plurality of pixel and/or voxel intensities. A higher first order median value may correlate to denser tissues, while a lower first order median value may correlate to low-density regions and/or tissues.

Machine learning model 116 may be trained to generate a future risk of vertebra fracture based on one or more radiomic features 212 of voxel volume, shape elongation, shape flatness, maximum 2D diameter row, shape sphericity, first order 10^{th} percentile, mean absolute deviation, first order mean, and first order median. In some embodiments, radiomic features 212 may be weighted. “Weighted” refers to a variable assigned a value corresponding to a relative importance of that variable. Weightage may be on a scale of about 0 to about 1, with 1 being a highest importance. In some embodiments, each weight of a variable, such as radiomic features 212, may sum to a total of 1. For instance, voxel volume may be assigned a weight of about 0.3, shape elongation may be assigned a weight of about 0.2, shape flatness may be assigned a weight of about 0.1, maximum 2D diameter row may be assigned a weight of about 0.1, row sphericity may be assigned a weight of about 0.1, first order 10^{th} percentile may be assigned a weight of about 0.05, mean absolute deviation may be assigned a weight of about 0.05, first order mean may be assigned a weight of about 0.05, and first order median may be assigned a weight of about 0.05.

In some embodiments, machine learning model 116 may be trained to input one or more clinical features. A “clinical feature” as used in this disclosure refers to information pertaining to a symptom and/or condition of a patient. Clinical features may include, but are not limited to, symptoms, lab results, demographic information, Eastern Cooperative Oncology Group (ECOG) scores, presence of vertebrae fracture, absence of vertebrae fracture, or other data. For instance, clinical features may include histology, age, body mass index (BMI), or other data. Machine learning model 116 may be trained to identify correlations between one or more clinical features and risks of future vertebra fracture. For instance, and without limitation, an older age may be correlated to a higher risk of vertebrae fracture within a given timeline, such as, but not limited to, a day, week, month, year, or longer than a year. Machine learning model 116 may be trained to input both radiomic features 212 and clinical features to determine a prediction of a risk of future fracture of one or more vertebra. In some embodiments, machine learning model 116 may be trained to exclusively utilize radiomic features 212 to determine a prediction of a risk of future fracture of one or more vertebra.

### Heterogenous Cohorts

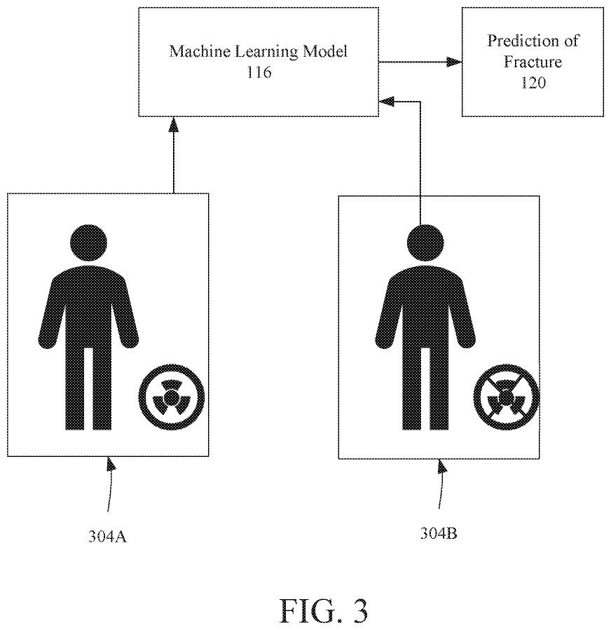

Referring now to FIG. 3, an illustration of a machine learning model with a cohort of patients is presented. Machine learning model 116 may be trained on training data that has a heterogeneous arrangement of two or more patients. “Heterogenous” as used in this disclosure refers to a mixing of two or more types of data. For instance, cohort of patients 300 may include, but is not limited to, toddlers (e.g., less than 3 years of age), young children (e.g., between 4 and 10 years of age), adolescents (e.g., between 10 and 19 years of age), young adults (e.g., between 19 and 24 years of age), adults, geriatric individuals, high BMI individuals, low BMI individuals, patients with previous vertebra fracture, patients without previous vertebra fracture, two or more races, two or more ethnicities, or any other combination of patients. In some embodiments, a cohort of patients may include radiated patient 304A. A “radiated patient” as used in this disclosure refers to a patient that previously had been exposed to radiation. Radiation exposure may include, but is not limited to, conventional radiation, stereotactic radiation, X-rays, or other forms of radiation. In some embodiments, dosimetric data may be obtained from radiated patient 304A and used by machine learning model 116 in generating prediction of fracture 120. “Dosimetric data” as used in this disclosure refers to any information pertaining to radiation applied to an individual. For instance and without limitation, dosimetric data may include radiation types, radiation amounts, radiation volumes, or other data. Dosimetric data may include a mean dose to the vertebrae, a maximum dose to the vertebrae, a volume of vertebrae receiving a dose, or other data. Dosimetric data may include a volume receiving dose value (Vx) of, but not limited to, V20, V20 V30, V40, V50, V60, V70, V80, V90, V100, or greater than V100. A Vx level may indicate how much of a specific anatomical structure receives at least a certain amount of radiation dose. A radiation dose may be Gray (Gy) radiation. For instance, a Vx level of about V30 may indicate that about 45% of the spinal cord volume received at least 30 Gy of radiation. Dosimetric data may correlate to vertebra data. For instance, higher doses of radiation and/or increased volume of radiation coverage may damage bone cells, such as, but not limited to, osteoblasts and/or osteoclasts, which may reduce bone density. A reduction in bone density may lead to an increase in probability of vertebral fracture. For instance and without limitation, high radiation doses, such as but not limited to greater than about V30, may lead to radiation-induced bone loss, microfractures, and/or an increased probability of vertebral compression fractures. In some embodiments, machine learning model 116 may be trained to correlate dosimetric data with one or more vertebrae parameters of a patient.

A cohort of patients may include at least one radiation-naïve patient 304B. A “radiation-naïve patient” as used in this disclosure refers to an individual that has not received vertebral radiation. Radiation-naïve patient 304B may not have dosimetric data. Machine learning model 116 may be trained with a plurality of radiation-naïve patients 304B that may all be absent in dosimetric data. Machine learning model 116 may be trained to input data absent dosimetric data and generate one or more predictions of fracture 120. For instance, machine learning model 116 may be trained with training data correlating one or more radiometric features to one or more predictions of fracture 120, free of dosimetric data. In some embodiments, machine learning model 116 may be trained with training data of a plurality of patients having at least one radiation-naïve patient. Machine learning model 116 may be trained to calculate prediction of fracture 120 across a wide variety of patients, such as both radiation-naïve, radiated, prior history of vertebral fracture, no history of vertebral fracture, kids, teenagers, young adults, adults, geriatric individuals, patients with tumor, patients without tumor, or other patient populations.

### Computer-Implemented Methods

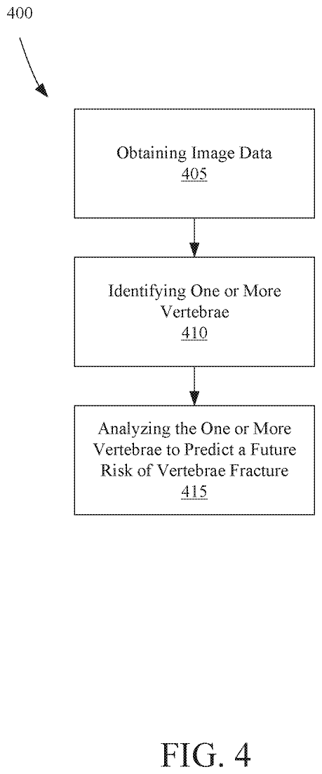

Referring now to FIG. 4, a flowchart of a computer-implemented method 400 of predicting risks of future vertebral fracture is presented. At step 405, method 400 includes obtaining image data. Image data may be obtained at a computing device via user input and/or external computing devices. In some embodiments, image data may include CT images, X-ray images, or other images. Image data may be exclusively CT images, in some embodiments.

At step 410, method 400 includes identifying one or more vertebrae. Vertebrae may be identified within image data. In some embodiments, vertebrae may be identified through a machine learning model, such as, but not limited to, a classifier, computer-vision model, or other forms of models. In particular embodiments, the machine learning model is a neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks). A machine learning model may be trained to input image data and output identifications of vertebral bodies within image data. Identification may determine one or more vertebral structures within image data. In some embodiments, identification may include annotating and/or highlighting one or more vertebral structures within image data.

At step 415, method 400 includes analyzing one or more vertebrae to predict a future risk of vertebrae fracture through a machine learning model. A machine learning model may be trained on a cohort of a population including at least one radiation-naive patient. Analysis may include processing features of one or more images. Features may include radiomic features. Radiomic features may correlate to a structural integrity of one or more vertebrae. Radiomic features may include shape-based, intensity-based, and/or texture-based features, without limitation. In some embodiments, features may be generated through one or more filters. Filters may include, but are not limited to, wavelet, square, exponential, logarithmic, or other forms of filters as described herein. In some embodiments, a single filter may be applied. In other embodiments, two or more filters may be applied. Application of a filter to an image may assist in a machine learning model extracting a feature, such as a radiomic feature. For instance, filters may increase contrast, edge detection, decrease overly bright spots, increase overly dark spots, or modify image data in other ways described herein. A machine learning model may input a plurality of voxels and/or pixels. A machine learning model may analyze one or more voxels and/or pixels to identify vertebrae height, width, length, volume, structural integrity, angle, or other metrics. In some embodiments, features may be selected through a feature selection process. A feature selection process may include a classifier, recursive feature elimination (RFE), linear regression, or other forms of feature selection. A feature selection process may select a top 50, top 40, top 30, top 20, top 10, or top 5 number of features to use for predicting future risks of vertebral fracture.

### Machine Learning Models

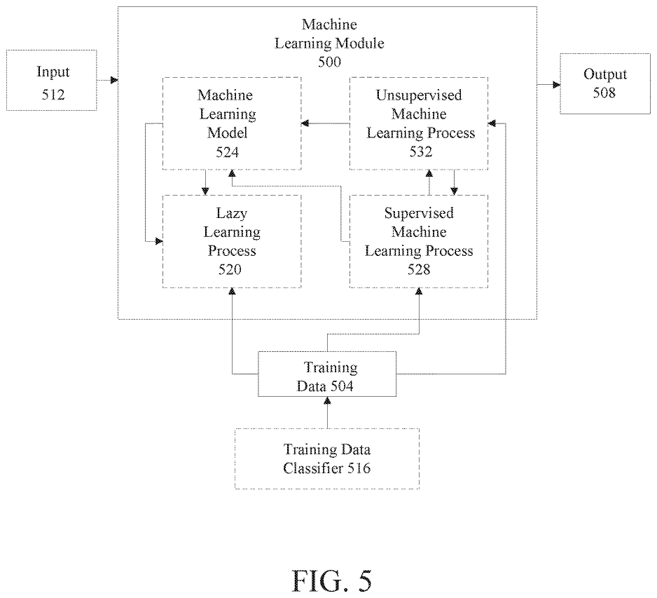

Referring now to FIG. 5, Referring to FIG. 5, an exemplary machine learning module 500 may perform machine learning process(es) and may be configured to perform various determinations, calculations, processes and the like as described herein using one or more machine learning processes.

In various embodiments, risk prediction models disclosed herein achieve a performance metric. Example performance metrics include an area under the curve (AUC-ROC) of a receiver operating curve, a positive predictive value, and/or a negative predictive value. In various embodiments, risk prediction models disclosed herein exhibit an AUC-ROC value of at least 0.5. In various embodiments, risk prediction models disclosed herein exhibit an AUC-ROC value of at least 0.6. In various embodiments, risk prediction models disclosed herein exhibit an AUC-ROC value of at least 0.7. In various embodiments, risk prediction models disclosed herein exhibit an AUC-ROC value of at least 0.8. In various embodiments, risk prediction models disclosed herein exhibit an AUC-ROC value of at least 0.9. In various embodiments, risk prediction models disclosed herein exhibit an AUC-ROC value of at least 0.95. In various embodiments, risk prediction models disclosed herein exhibit an AUC-ROC value of at least 0.99. In various embodiments, risk prediction models disclosed herein exhibit an AUC-ROC value of at least 0.51, at least 0.52, at least 0.53, at least 0.54, at least 0.55, at least 0.56, at least 0.57, at least 0.58, at least 0.59, at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, at least 0.74, at least 0.75, at least 0.76, at least 0.77, at least 0.78, at least 0.79, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, or at least 0.99.

In some embodiments, risk prediction models disclosed herein may achieve a sensitivity of, but not limited to, at least 0.51, at least 0.52, at least 0.53, at least 0.54, at least 0.55, at least 0.56, at least 0.57, at least 0.58, at least 0.59, at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, at least 0.74, at least 0.75, at least 0.76, at least 0.77, at least 0.78, at least 0.79, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, or at least 0.99.

In some embodiments, risk prediction models disclosed herein may achieve a specificity of, but not limited to, at least 0.51, at least 0.52, at least 0.53, at least 0.54, at least 0.55, at least 0.56, at least 0.57, at least 0.58, at least 0.59, at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, at least 0.74, at least 0.75, at least 0.76, at least 0.77, at least 0.78, at least 0.79, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, or at least 0.99.

In some embodiments, risk prediction models disclosed herein may achieve a 95% confidence interval (CI) of, but not limited to, at least 0.51, at least 0.52, at least 0.53, at least 0.54, at least 0.55, at least 0.56, at least 0.57, at least 0.58, at least 0.59, at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, at least 0.74, at least 0.75, at least 0.76, at least 0.77, at least 0.78, at least 0.79, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, or at least 0.99.

Machine learning module 500 may utilize training data 504. For instance, and without limitation, training data 504 may include a plurality of data entries, each entry representing a set of data elements that were recorded, received, and/or generated together. Training data 504 may include data elements that may be correlated by shared existence in a given data entry, by proximity in a given data entry, or the like. Multiple data entries in training data 504 may demonstrate one or more trends in correlations between categories of data elements. For instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 504 according to various correlations. Correlations may indicate causative and/or predictive links between categories of data elements, which may be modeled as relationships such as mathematical relationships by machine learning processes as described in further detail below. Training data 504 may be formatted and/or organized by categories of data elements. Training data 504 may, for instance, be organized by associating data elements with one or more descriptors corresponding to categories of data elements. As a non-limiting example, training data 504 may include data entered in standardized forms by one or more individuals, such that entry of a given data element in a given field in a form may be mapped to one or more descriptors of categories. Elements in training data 504 may be linked to descriptors of categories by tags, tokens, or other data elements. Training data 504 may be provided in fixed-length formats, formats linking positions of data to categories such as comma-separated value (CSV) formats and/or self-describing formats. Self-describing formats may include, without limitation, extensible markup language (XML), JavaScript Object Notation (JSON), or the like, which may enable processes or devices to detect categories of data.

With continued reference to refer to FIG. 5, training data 504 may include one or more elements that are not categorized. Uncategorized data of training data 504 may include data that may not be formatted or containing descriptors for some elements of data. In some embodiments, machine learning algorithms and/or other processes may sort training data 504 according to one or more categorizations. Machine learning algorithms may sort training data 504 using, for instance, natural language processing algorithms, tokenization, detection of correlated values in raw data and the like. In some embodiments, categories of training data 504 may be generated using correlation and/or other processing algorithms. As a non-limiting example, in a body of text, phrases making up a number “n” of compound words, such as nouns modified by other nouns, may be identified according to a statistically significant prevalence of n-grams containing such words in a particular order. For instance, an n-gram may be categorized as an element of language such as a “word” to be tracked similarly to single words, which may generate a new category as a result of statistical analysis. In a data entry including some textual data, a person's name may be identified by reference to a list, dictionary, or other compendium of terms, permitting ad-hoc categorization by machine learning algorithms, and/or automated association of data in the data entry with descriptors or into a given format. The ability to categorize data entries in an automated fashion may enable the same training data 504 to be made applicable for two or more distinct machine learning algorithms as described in further detail below. Training data 504 used by machine learning module 500 may correlate any input data as described in this disclosure to any output data as described in this disclosure, without limitation.

Further referring to FIG. 5, training data 504 may be filtered, sorted, and/or selected using one or more supervised and/or unsupervised machine learning processes and/or models as described in further detail below. In some embodiments, training data 504 may be classified using training data classifier 516. Training data classifier 516 may include a classifier. A “classifier” as used in this disclosure is a machine-learning model that sorts inputs into one or more categories. Training data classifier 516 may utilize a mathematical model, an artificial neural network, or a program generated by a machine learning algorithm. A machine learning algorithm of training data classifier 516 may include a classification algorithm. A “classification algorithm” as used herein is one or more computer processes that generate a classifier from training data. A classification algorithm may sort inputs into categories and/or bins of data. A classification algorithm may output categories of data and/or labels associated with the data. A classifier may be configured to output a datum that labels or otherwise identifies a set of data that may be clustered together. Machine learning module 500 may generate a classifier, such as training data classifier 516 using a classification algorithm. Classification may be performed using, without limitation, linear classifiers such as without limitation logistic regression and/or naive Bayes classifiers, nearest neighbor classifiers such ask-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data to one or more types of vertebrae and/or probability of future risks of vertebral fractures.

Still referring to FIG. 5, machine learning module 500 may be configured to perform a lazy-learning process 520 which may include a “lazy loading” or “call-when-needed” process and/or protocol. A “lazy-learning process” may include a process in which machine learning is performed upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 504. Heuristic may include selecting some number of highest-ranking associations and/or training data 504 elements. Lazy learning may implement any suitable lazy learning algorithm, including without limitation a K-nearest neighbors algorithm, a lazy naive Bayes algorithm, or the like. Persons skilled in the art, upon reviewing the entirety of this disclosure, will be aware of various lazy-learning algorithms that may be applied to generate outputs as described herein, including lazy learning applications of machine-learning algorithms as described in further detail below.

Still referring to FIG. 5, machine learning processes as described herein may be used to generate machine learning models 524. A “machine learning model” as used herein is a mathematical and/or algorithmic representation of a relationship between inputs and outputs, as generated using any machine learning process including without limitation any process as described above, and stored in memory. For instance, an input may be sent to machine learning model 524, which once created, may generate an output as a function of a relationship that was derived. For instance, and without limitation, a linear regression model, generated using a linear regression algorithm, may compute a linear combination of input data using coefficients derived during machine learning processes to calculate an output. As a further non-limiting example, machine learning model 524 may be generated by creating an artificial neural network, such as a convolutional neural network comprising an input layer of nodes, one or more intermediate layers, and an output layer of nodes. Connections between nodes may be created via the process of “training” the network, in which elements from a training data 504 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes.

Still referring to FIG. 5, machine learning algorithms may include supervised machine learning process 528. A “supervised machine learning process” as used herein is one or more algorithms that receive labelled input data and generate outputs according to the labelled input data. For instance, supervised machine learning process 528 may include features as described above as inputs, predictions of future risks of vertebrae fracture as outputs, and a scoring function representing a desired form of relationship to be detected between inputs and outputs. A scoring function may maximize a probability that a given input and/or combination of elements inputs is associated with a given output to minimize a probability that a given input is not associated with a given output. A scoring function may be expressed as a risk function representing an “expected loss” of an algorithm relating inputs to outputs, where loss is computed as an error function representing a degree to which a prediction generated by the relation is incorrect when compared to a given input-output pair provided in training data 504. Persons skilled in the art, upon reviewing the entirety of this disclosure, will be aware of various possible variations of at least a supervised machine learning process 528 that may be used to determine relation between inputs and outputs. Supervised machine learning processes may include classification algorithms as described herein.

Further referring to FIG. 5, machine learning processes may include unsupervised machine learning processes 532. An “unsupervised machine learning process” as used herein is a process that calculates relationships in one or more datasets without labelled training data. Unsupervised machine learning process 532 may be free to discover any structure, relationship, and/or correlation provided in training data 504. Unsupervised machine learning process 532 may not require a response variable. Unsupervised machine learning process 532 may calculate patterns, inferences, correlations, and the like between two or more variables of training data 504. In some embodiments, unsupervised machine learning process 532 may determine a degree of correlation between two or more elements of training data 504.

Still referring to FIG. 5, machine learning module 500 may be designed and configured to create a machine learning model 524 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of I divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model. Linear regression models may be generalized in an embodiment to polynomial regression models, whereby a polynomial equation (e.g. a quadratic, cubic or higher-order equation) providing a best predicted output/actual output fit is sought. Similar methods to those described above may be applied to minimize error functions, according to some embodiments. In some embodiments, machine learning model 524 may utilize one or more encoders and/or decoders, transformer architectures, attention mechanisms, self-attention mechanisms, multi-head attention, masked multi-head attention, token biasing, probability biasing, feed forward layers, positional encoding, recurrent decoders, or any other processes that may be implemented.

Continuing to refer to FIG. 5, machine learning algorithms may include, without limitation, linear discriminant analysis. Machine learning algorithm may include quadratic discriminate analysis. Machine learning algorithms may include kernel ridge regression. Machine learning algorithms may include support vector machines, including without limitation support vector classification-based regression processes. Machine learning algorithms may include stochastic gradient descent algorithms, including classification and regression algorithms based on stochastic gradient descent. Machine learning algorithms may include nearest neighbors algorithms. Machine learning algorithms may include various forms of latent space regularization such as variational regularization. Machine learning algorithms may include Gaussian processes, such as Gaussian Process Regression. Machine learning algorithms may include cross-decomposition algorithms, including partial least squares and/or canonical correlation analysis. Machine learning algorithms may include naive Bayes methods. Machine learning algorithms may include algorithms based on decision trees, such as decision tree classification or regression algorithms. Machine learning algorithms may include ensemble methods such as bagging meta-estimator, forest of randomized tress, AdaBoost, gradient tree boosting, and/or voting classifier methods. Machine learning algorithms may include neural net algorithms, including convolutional neural net processes.

### Diseases

Methods and systems described herein may treat one or more diseases. Diseases may include, but are not limited to, degenerative disc disease (DDD), spinal stenosis, spondylosis, facet joint osteoarthritis, spondylolisthesis, vertebral fracture, spinal cord injury (SCI), osteosarcoma, chordoma, metastatic tumors, scoliosis, kyphosis, lordosis, or other diseases. Methods and systems described herein treat osteoporotic insufficiency and/or disease states resulting from vertebral fracture. For instance, methods and systems described herein may treat spinal cord compression, nerve root and/or nerve compression, or any other disease state that may occur due to vertebral insufficiency and/or fracture.

### Guided Therapy

In some embodiments, prediction of risks of vertebrae fractures or other data described herein may be presented to a medical professional. A medical professional may incorporate predictions of risks of vertebrae fractures in various forms of therapy. For instance, if a prediction of a risk of vertebrae fracture is high, a therapy may include addressing bone health by providing one or more pharmacologic therapies, such as, but not limited to, Bisphosphonates, Denosumab, Teriparatide, Calcium, Vitamin D, or other therapies. A therapy may include monitoring bone density to assess further bone degeneration. A therapy may include providing a brace, limiting high-impact activities, or other therapies. A therapy may include surgical prevention, such as, but not limited to, prophylactic vertebroplasty or kyphoplasty. A therapy may include spinal instrumentation, such as preemptive fixation or fusion, without limitation. A therapy may include early, preventative, and/or prophylactic spinal radiation. If a future risk of vertebra fracture is about 0%, a medical professional may monitor potential development of vertebral degeneration. In some embodiments, if a future risk of vertebra fracture is about low, such as lower than about 15%, a medical professional may exclude a patient with a low future risk of vertebra fracture from Bisphosphonates, Denosumab, or other medication treatments. A medical professional may mark a patient having a low future risk of vertebra fracture as being excluded being offered prophylactic cement augmentation and/or radiation or other follow up treatments. Medical professionals may include, but are not limited to, primary care physicians, emergency room (ER) doctors, medical oncology, radiation oncology, interventionists, neurosurgeons, diagnostic radiation, or other medical professionals.

### Computing Environments

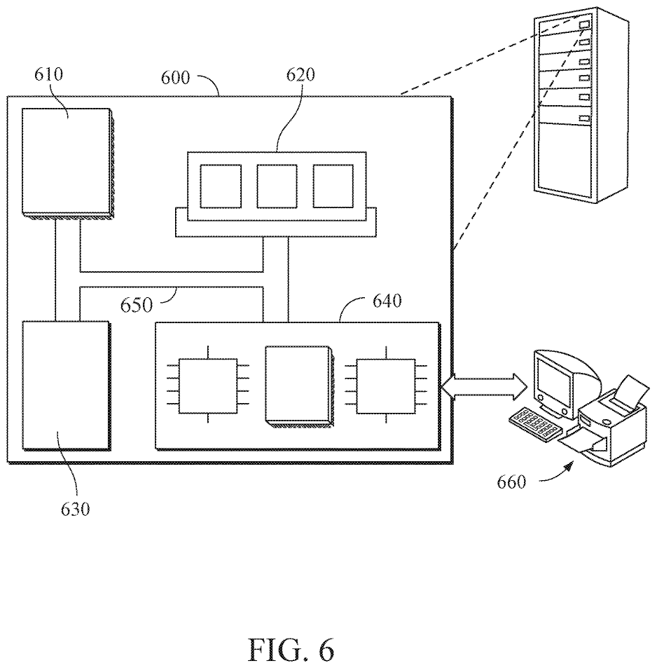

Referring now to FIG. 6 is a block diagram of an example computer system 600 that may be used in implementing the technology described in this document. General-purpose computers, network appliances, mobile devices, or other electronic systems may also include at least portions of the system 600. The system 600 includes a processor 610, a memory 620, a storage device 630, and an input/output device 640. The apparatus may include disk storage and/or internal memory, each of which may be communicatively connected to each other. The apparatus 100 may include a processor 610. The processor 610 may enable both generic operating system (OS) functionality and/or application operations. In some embodiments, the processor 610 and the memory 620 may be communicatively connected. As used in this disclosure, “communicatively connected” means connected by way of a connection, attachment, or linkage between two or more elements which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device.

Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure. In some embodiments, the processor 610 may include any computing device as described in this disclosure, including without limitation a microcontroller, microprocessor, digital signal processor (DSP) and/or system on a chip (SoC) as described in this disclosure. The processor 610 may include, be included in, and/or communicate with a mobile device such as a mobile telephone or smartphone. The processor 610 may include a single computing device operating independently, or may include two or more computing device operating in concert, in parallel, sequentially or the like. Two or more computing devices may be included together in a single computing device or in two or more computing devices. The processor 610 may interface or communicate with one or more additional devices as described below in further detail via a network interface device. Network interface device may be utilized for connecting the processor 610 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device.

The processor 610 may include but is not limited to, for example, a computing device or cluster of computing devices in a first location and a second computing device or cluster of computing devices in a second location. The processor 610 may include one or more computing devices dedicated to data storage, security, distribution of traffic for load balancing, and the like. The processor 610 may distribute one or more computing tasks as described below across a plurality of computing devices, which may operate in parallel, in series, redundantly, or in any other manner used for distribution of tasks or memory between computing devices. The processor 610 may be implemented using a “shared nothing” architecture in which data is cached at the worker, in an embodiment, this may enable scalability of system 600 and/or processor 610.

With continued reference to FIG. 6, processor 610 and/or a computing device may be designed and/or configured by memory 620 to perform any method, method step, or sequence of method steps in any embodiment described in this disclosure, in any order and with any degree of repetition. For instance, the processor 610 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. The processor 610 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Each of the components 610, 620, 630, and 640 may be interconnected, for example, using a system bus 650. The processor 610 is capable of processing instructions for execution within the system 600. In some implementations, the processor 610 is a single-threaded processor. In some implementations, the processor 610 is a multi-threaded processor. In some implementations, the processor 610 is a programmable (or reprogrammable) general purpose microprocessor or microcontroller. The processor 610 is capable of processing instructions stored in the memory 620 or on the storage device 630.

The memory 620 stores information within the system 600. In some implementations, the memory 620 is a non-transitory computer-readable medium. In some implementations, the memory 620 is a volatile memory unit. In some implementations, the memory 620 is a non-volatile memory unit.

The storage device 630 is capable of providing mass storage for the system 600. In some implementations, the storage device 630 is a non-transitory computer-readable medium. In various different implementations, the storage device 630 may include, for example, a hard disk device, an optical disk device, a solid-date drive, a flash drive, or some other large capacity storage device. For example, the storage device may store long-term data (e.g., database data, file system data, etc.). The input/output device 640 provides input/output operations for the system 600. In some implementations, the input/output device 640 may include one or more network interface devices, e.g., an Ethernet card, a serial communication device, e.g., an RS-232 port, and/or a wireless interface device, e.g., an 802.11 card, a 3G wireless modem, or a 4G/5G wireless modem. In some implementations, the input/output device may include driver devices configured to receive input data and send output data to other input/output devices, e.g., keyboard, printer and display devices 660. In some examples, mobile computing devices, mobile communication devices, and other devices may be used.

### EXAMPLES

### Example 1: Machine Learning Model Successfully Predicts Likelihood of Vertebrae Fracture

TABLE 1

| Characteristic | Value |
|---|---|
| Total patients | 164 |
| Total vertebrae | 977 |
| Age, yrs | 63.2 (25.0-87.4) |
| Female | 465 (47.6%) |
| BMI, kg/m2 | 27.7 (12.8-47.4) |
| Primary histology | |
| Breast | 265 (27.1%) |
| Prostate | 207 (21.2%) |
| Multiple myeloma | 130 (13.3%) |
| Other | 113 (11.6%) |
| Renal | 94 (9.6%) |
| Colorectal | 73 (7.5%) |
| NSCLC | 63 (6.4%) |
| Melanoma | 30 (3.1%) |
| Unknown | 2 (0.2%) |
| High-dose steroid use | 214 (21.9%) |
| Bisphosphonate use | 562 (57.5%) |
| Osteoporosis | 550 (56.3%) |
| Systemic anticancer therapy | 922 (94.4%) |
| Vertebral level | |
| Thoracic | 714 (73.1%) |
| Lumbar | 263 (26.9%) |
| SINS | |
| Total score | 5.2 (1-14) |
| 0-6 (stable) | 677 (69.3%) |
| 7-12 (potentially unstable) | 293 (30.0%) |
| 13-18 (unstable) | 6 (0.6%) |
| Prior radiation | 251 (25.7%) |
| Stereotactic | 99 (39.4%) |
| Non-stereotactic | 152 (60.6%) |
| Pre-existing VCF | 179 (18.3%) |
| VCF, new or progressive (Outcome 1) | 123 (12.6%) |

NSCLC = non-small cell lung cancer. Values represent number of vertebral levels (%) or mean (range).

Table 1 illustrates various characteristics of a cohort of patients used to train models described herein. After Institutional Review Board approval (from each study site), the electronic medical records (EMR) from three academic hospitals were searched by ICD10 codes to include pathologic vertebral compression fractures between Jun. 1, 2013 and Mar. 1, 2022. These patients were then screened for having CT of index level(s) (thoracolumbar). Each study vertebra was required to have tumor infiltration as confirmed by 3D imaging of the spine as documented in radiology report and/or determined upon secondary review by a board-certified neurosurgeon. The time of first documentation (on 3D imaging) of new vertebral compression fracture (VCF) was identified. Then, the first CT prior (chronologically) to this outcome that captured the study vertebrae before the outcome was used for radiomic extraction. If no VCF occurred in the patient's history, then a CT that captured the study vertebra was used for which up to one year follow of outcome was documented.

The included vertebrae were categorized as non-fractured vertebral body or pre-existing fractured vertebral body. The vertebral bodies that developed new fracture and the pre-existing fractures that progressed, were grouped into outcome 1 (VCF). The vertebral bodies that did not fracture and the pre-existing fractures that did not progress, were grouped into outcome 0 (no VCF). VCF was defined as new height loss of vertebral body or further vertebral body height loss of a pre-existing compression fracture.

Exclusion criteria included patients with only cervical pathology, no tumor infiltrated vertebrae, compression fracture etiology other than tumor (e.g. insufficiency, infection) or unknown etiology, those with progression of local metastatic disease (progression of tumor growth within the studied vertebral levels) between initial CT and time of fracture/fracture progression or during the follow up period (if no VCF occurred). The CT DICOMs were extracted from the respective EMRs onto securely stored CDs, and then uploaded to a secure server for further management. After review on the server, the data were deidentified according to HIPAA/IRB standards and downloaded for offline management.

From the patients that met the above criteria, clinical data were collected. “Other” histologies were those representing less than 2% of all represented histologies. There were 2 (0.2%) vertebrae where the histology was unknown. “Prior radiation” included any radiation therapy where the study vertebra was the target. “Stereotactic” radiation (SR) included stereotactic body radiation therapy (SBRT) and stereotactic radiosurgery (SRS). “Non-stereotactic” included all other radiation modalities, primarily conventional. “High-dose steroids” were considered as 4 mg dexamethasone (or equivalent) per day for at least 1 month prior to VCF or final follow-up in the case of no VCF in the patient's history. Osteoporosis was determined by presence of diagnosis code (ICD10).

A total of 164 patients were included in the study, with varying numbers of thoracolumbar vertebrae from each patient, representing a total of 977 vertebrae. Each vertebra was treated as an individual instance and the characteristics of these vertebrae are provided in Table 1. On a per vertebrae analysis, the average age was 63.2 years (25.0-87.4). Approximately one half were female (47.6%). The average BMI was 27.7 (12.8-47.4). The most common histology was breast (27.1%), followed by prostate (21.2%), and multiple myeloma (13.3%). There were 2 vertebrae (0.2%) of unknown histology. A minority were subjected to high-dose steroids (21.9%), while approximately one-half were on bisphosphonates (57.5%), and the majority were on systemic anticancer medications (94.4%). Approximately one-half had been diagnosed with osteoporosis (56.3%). Of the 977 included thoracolumbar vertebrae, the majority were thoracic (73.1%). The average spinal instability neoplastic score (SINS) score for the vertebrae was 5.2 (1-14), with the majority being score 0-6 (69.3%). A total of 251 (25.7%) vertebrae had received radiation, with 99 (39%) of these being SR (SRS or SBRT). One-hundred-seventy-nine (18.3%) of the vertebrae had preexisting compression fractures. There were 123 (12.6%) vertebrae that developed VCF (outcome 1).

TABLE 2

| | Univariate | | | Multivariate | | |
|---|---|---|---|---|---|---|
| Feature | OR | 95% CI | p-value | OR | 95% CI | p-value |
| SINS score, | 1.21 | 1.12-1.30 | 2.16e−07 | 1.10 | 1.01-1.21 | 0.037 |
| total | | | | | | |
| BMI | 1.02 | 0.98-1.05 | 0.324 | 1.02 | 0.98-1.06 | 0.326 |
| Age | 0.99 | 0.97-1.00 | 0.1031 | 0.99 | 0.97-1.01 | 0.22 |
| Osteo- | 0.99 | 0.68-1.45 | 0.962 | 1.01 | 0.63-1.62 | 0.972 |
| porosis | | | | | | |
| −1 | | | | | | |
| None of the | 0.27 | 0.18-0.41 | 1.84e−10 | 0.67 | 0.38-1.18 | 0.158 |
| Prior 3-1 | | | | | | |
| HistoColon | 0.85 | 0.28-2.19 | 0.76 | 1.58 | 0.49-4.32 | 0.404 |
| HistoMe- | 0.83 | 0.13-3.04 | 0.808 | 1.14 | 0.17-4.53 | 0.871 |
| lanoma | | | | | | |
| HistoMM | 1.99 | 1.02-3.85 | 0.04 | 1.80 | 0.85-3.80 | 0.122 |
| Histo- | 1.69 | 0.67-3.88 | 0.235 | 2.27 | 0.82-5.83 | 0.099 |
| NSCLC | | | | | | |
| HistoOther | 3.83 | 2.07-7.17 | 2.03e−05 | 5.33 | 2.74-10.6 | 1.0e−06 |
| Histo- | 1.11 | 0.57-2.14 | 0.763 | 1.59 | 0.76-3.34 | 0.215 |
| Prostate | | | | | | |
| cancer | | | | | | |
| HistoRCC | 3.14 | 1.61-6.13 | 7.49e−4 | 3.73 | 1.79-7.79 | 4.2e−04 |
| HistoUn- | 2.46e7 | NA | 0.978 | 1.79 | NA | 0.977 |
| known | | | | | | |
| <50% | 6.75 | 4.36-10.4 | <2e−16 | 4.76 | 2.76-8.31 | 2.7e−08 |
| collapse −1 | | | | | | |

Recursive feature elimination (RFE) was performed individually on the radiomic feature set, individually on the clinical feature set (included all SINS components, SINS total score, and 9 clinical features listed in Table 1), and lastly on combined clinical and radiomics features. For the clinical feature set, the features selected by RFE and used to build the clinical-only models are show in Table 2.

CT DICOMs were downloaded from the secure server, and reviewed on 3D Slicer The DICOMs were then converted to NIFTI files. ITK-SNAP was used to open these source NIFTI files. Through ITK-SNAP, the entire vertebral bodies were manually segmented and each of these segmentations was saved as an individual mask alongside its source image. Subsequently, eight preprocessing filters were individually applied to the source images (wavelet, square, square root, logarithm, exponential, Laplacian of Gaussian, gradient, local binary pattern 3D) and all PyRadiomics feature classes were extracted from these images, as well original source images. The radiomic feature classes used can be subdivided into 3 major subclasses: 14 shape-based, 18 intensity-based, and 75 texture-based features for a total of 107 features extracted from the original images (sans pre-processing filters). After application of each aforementioned preprocessing filters, features were extracted from the resulting filtered image, resulting in a set of unique features per filtered image, totaling 2015 unique features. The resulting selected features from the radiomic feature set that were used to build the radiomic-only prediction model are shown in Table 3. Due to the large number of radiomic features, extra feature selection measures were required to reduce the dimensionality of data. Variables with zero variation were first excluded. Then, highly correlated features based on Pearson's correlation coefficient >0.9 were excluded. Wilcoxon rank sum tests were then performed to select predictors that were significantly associated (p<0.01) with fracture development or progression. All non-significant variables were discarded. After RFE, the selected variables chosen were evaluated for odds-ratios and significance values with logistic regression.

Prior to RFE, 79 features were excluded due to zero variance and 1487 variables were then excluded due to redundancy in information as they were highly correlated. From the remaining 527 variables, 88 significantly varied with outcome and were carried forward to RFE. RFE yielded 19 top radiomic features that maximized area under the receiver operating characteristic curve (AUC-ROC).

TABLE 3

| | Univariate | | | Multivariate | | |
|---|---|---|---|---|---|---|
| | | | p- | | | p- |
| Feature | OR | 95% CI | value | OR | 95% CI | value |
| lbp-3D-k_gldm_DependenceVariance | 1.05 | 1.01-1.10 | 0.02 | 1.10 | 1.03-1.18 | 0.03e−1 |
| lbp-3D-m1_firstorder_RootMeanSquared | 1.53 | 0.98-2.37 | 0.06 | 0.68 | 1.28e−01-3.61 | 0.65 |
| lbp-3D-k_firstorder_Maximum | 1.17 | 1.00-1.35 | 0.04 | 1.00 | 0.80-1.24 | 0.99 |
| wavelet-LHL_ngtdm_Strength | 1.05 | 0.85-1.24 | 0.62 | 1.23 | 9.84e−01-1.60 | 0.10 |
| wavelet- | 16.50 | 4.74-65.95 | 3.23e−05 | 0.21 | 8.70e−03-4.00 | 0.32 |
| LLL_gldm_LargeDependenceLowGrayLevelEmphasis | | | | | | |
| lbp-3D-m2_girlm_RunLengthNonUniformity | 1.00 | 1.00-1.00 | 0.03 | 1.00 | 9.99e−01-1.000 | 0.31 |
| original_gldm_LargeDependenceLowGrayL | 1.53 | 1.29-1.86 | 1.19e−06 | 1.66 | 1.15-2.44 | 0.01 |
| evelEmphasis | | | | | | |
| square_glszm_ZoneVariance | 1.00 | 1.00-1.00 | 0.01 | 1.00 | 1.00-1.00 | 0.54 |
| logarithm_gldm_DependenceEntropy | 3.73 | 2.02-6.92 | 2.62e−05 | 9.74 | 2.55-38.96 | 0.01e−1 |
| original_gldm_LargeDependenceHighGrayLevelEmphasis | 1.00 | 1.00-1.00 | 0.74 | 1.00 | 1.00-1.00 | 0.16 |

Lbp-3D=local binary pattern-3 dimensional. LHL=low, high, low (wavelet). LLL=low, low, low (wavelet). Square=square filter. Logarithm=logarithm filter. Gldm=gray level dependence matrix. Ngtdm=neighboring gray tone difference matrix. Glrlm=gray level run length matrix. Glszm=gray level size zone matrix.

To create a combined clinical and radiomic feature set, the 7 clinical features (Table 2) and 19 radiomic features selected in aforementioned RFE were combined to create a clinical/radiomic set of 26 total features. Then, a second round of RFE was performed on this set, yielding 25 features (6 clinical and 19 radiomic) for the combined clinical and radiomic prediction models (Table 4). The 19 radiomic features retained by RFE for the combined clinical-radiomic models, are the same 19 features used in the radiomic-only model. The top 25 features by importance from RFE were used for the combined clinical-radiomic models.

The resulting selected features from the combined clinical and radiomic features set to build the clinical-radiomic models are shown in Table 4. These resulting features from the combined clinical and radiomic feature set were used to build the combine clinical and radiomic model. No feature selection was performed for the SINS model (all SINS components and total score were used).

TABLE 4

| Feature | OR | 95% CI | p-value |
|---|---|---|---|
| <50% collapse | 4.22 | 2.30-7.81 | 3.63e−06 |
| HistoOther | 5.76 | 2.68-12.69 | 9.22e−06 |
| HistoRCC | 3.56 | 1.57-8.12 | 2.4e−03 |
| HistoProstate Cancer | 1.38 | 0.63-3.04 | 0.43 |
| HistoMM | 1.62 | 0.96-3.76 | 0.26 |
| HistoNSCLC | 1.11 | 0.35-3.22 | 0.85 |
| HistoColon | 1.14 | 0.33-3.38 | 0.82 |
| HistoMelanoma | 2.05 | 0.26-9.82 | 0.42 |
| HistoUnknown | 2.52e+04 | NA | 0.99 |
| Age | 1.01 | 0.98-1.03 | 0.67 |
| BMI | 1.01 | 0.97-1.05 | 0.60 |
| None.of.the.prior 3 | 0.45 | 0.23-0.88 | 0.02 |
| SINS_score | 1.07 | 0.97-1.19 | 0.19 |
| LoG_Sigma4_glszm_ZoneVariance | 1.01 | 0.96-1.05 | 0.73 |
| original_gldm_LargeDependenceLowGrayLevelEmphasis | 1.88 | 1.17-3.01 | 0.01 |
| square_glszm_ZoneVariance | 1.00 | 1.00-1.00 | 0.82 |
| wavelet- | 0.08 | 1.24e−03-4.99 | 0.24 |
| LLL_gldm_LargeDependenceLowGrayLevelEmphasis | | | |
| logarithm_glrlm_RunEntropy | 0.75 | 0.19-2.84 | 0.67 |

LoG=Laplacian of Gaussian. LLL=low, low, low (wavelet). Square=square filter. Logarithm=logarithm filter. Gldm=gray level dependence matrix. Glrlm=gray level run length matrix. Glszm=gray level size zone matrix.

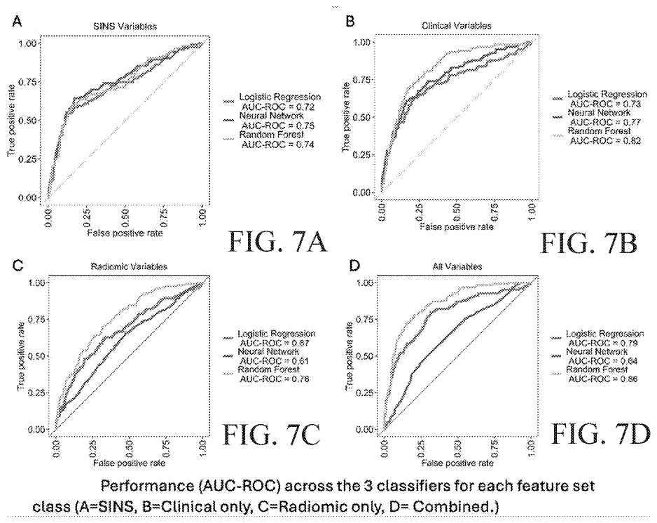

Referring now to FIGS. 7A-D, graphs illustrating model performance are presented. FIG. 7A illustrates performance using SINS variables. FIG. 7B illustrates performance using clinical variables. FIG. 7C illustrates performance using radiomic variables. FIG. 7D illustrates performance using all variables.

Using a random forest classifier, recursive feature elimination (RFE) was conducted to identify the smallest subset of predictors that could maximize the area under the receiver operating characteristic curve (AUC-ROC). This was performed for the clinical features individually, the radiomic features only, and on combined clinical and radiomic features to create the clinical-only models, radiomic-only models, and combined models, respectively. For each RFE, data was fed to the model using 5-fold cross-validation. The AUC-ROC curve graphs sensitivity and specificity at different classification thresholds, visually displaying a model's differentiating capability. AUC-ROC measures the area under that curve with a 0.5 AUC-ROC indicating random guessing and 1.0 AUC-ROC indicating a perfect classifier. Models that utilized both clinical and radiomic features underwent a second step of recursive feature elimination.

Three algorithms with tunable hyperparameters were used to build final classifiers for each feature set (SINS, clinical, radiomics, clinical plus radiomics): random forest, single hidden layer artificial neural network (ANN), and logistic regression. Selected features were fed to each algorithm using 10-fold cross-validation. An AUC-ROC curve was computed, and AUC-ROC was calculated. Sensitivity and specificity were optimized by choosing a threshold that maximized informedness, a measure derived from Youden's J statistic that conveys the probability that an estimator will make an informed prediction. Results of each of graphs A-D are depicted below in Table 5.

TABLE 5

| Variables | Model | AUC-ROC | 95% CI | Sensitivity | 95% CI | Specificity | 95% CI |
|---|---|---|---|---|---|---|---|
| Clinical | Logistic | 0.73 | 0.68-0.78 | 0.63 | 0.54-0.71 | 0.79 | 0.76-0.81 |
| | Regression | | | | | | |
| | ANN | 0.77 | 0.72-0.82 | 0.61 | 0.52-0.69 | 0.83 | 0.81-0.86 |
| | Random | 0.82 | 0.77-0.87 | 0.69 | 0.6-0.77 | 0.83 | 0.8-0.85 |
| | Forest | | | | | | |
| SINS | Logistic | 0.72 | 0.67-0.77 | 0.55 | 0.46-0.63 | 0.88 | 0.85-0.9 |
| | Regression | | | | | | |
| | ANN | 0.75 | 0.7-0.8 | 0.64 | 0.55-0.72 | 0.83 | 0.8-0.85 |
| | Random | 0.74 | 0.69-0.79 | 0.60 | 0.51-0.68 | 0.84 | 0.81-0.86 |
| | Forest | | | | | | |
| Radiomic | Logistic | 0.67 | 0.62-0.72 | 0.63 | 0.54-0.71 | 0.67 | 0.64-0.7 |
| | Regression | | | | | | |
| | ANN | 0.61 | 0.55-0.67 | 0.66 | 0.57-0.74 | 0.53 | 0.49-0.56 |
| | Random | 0.76 | 0.71-0.81 | 0.71 | 0.62-0.78 | 0.67 | 0.64-0.7 |
| | Forest | | | | | | |
| Combined | Logistic | 0.79 | 0.74-0.84 | 0.80 | 0.72-0.86 | 0.68 | 0.65-0.71 |
| | Regression | | | | | | |
| | ANN | 0.64 | 0.58-0.7 | 0.76 | 0.67-0.82 | 0.45 | 0.42-0.49 |
| | Random | 0.86 | 0.82-0.9 | 0.78 | 0.70-0.84 | 0.80 | 0.77-0.82 |
| | Forest | | | | | | |

Among all variable cohorts, the SINS group performed the worst with AUC-ROC ranging from 0.72-0.75 (FIG. 7). The purely clinical models (AUC-ROC: 0.73-0.82) performed approximately equal to the purely radiomic models (AUC-ROC: 0.67-0.76) (FIG. 7). Combining clinical and radiomic features resulted in the overall best performing classifiers, with AUC-ROC 0.64-0.86 (FIG. 7). Specifically, the random forest model using the combined clinical and radiomic features yielded the highest performance of AUC-ROC 0.86 (CI 0.82-0.9) (Table 5). This performance was significantly higher than the best SINS-alone model (AUC-ROC=0.74, CI 0.70-0.80) and outperformed the clinical-only model, but did not reach statistical significance (AUC-ROC=0.82, CI 0.77-0.87).

Within 1 year from initial CT, 123 of 977 vertebrae developed VBF. Selected clinical features included SINS score, SINS components <50% collapse, SINS “none of prior 3”, histology, age, and BMI. Of the 2015 radiomic features, RFE selected 19 to be used in the pure radiomic model and the combined clinical and radiomic model. The best-performing model was a random-forest classifier using both clinical and radiomic features, demonstrating an area under the receiver operating characteristic (ROC) curve (AUC-ROC) of 0.86 (CI 0.82-0.9), sensitivity 0.78 (CI 0.70-0.84), and specificity 0.80 (0.77-0.82). This performance was significantly higher than the best SINS-alone model (AUC-ROC=0.74, CI 0.70-0.80) and outperformed the clinical-only model but did not reach statistical significance (AUC-ROC=0.82, CI 0.77-0.87).

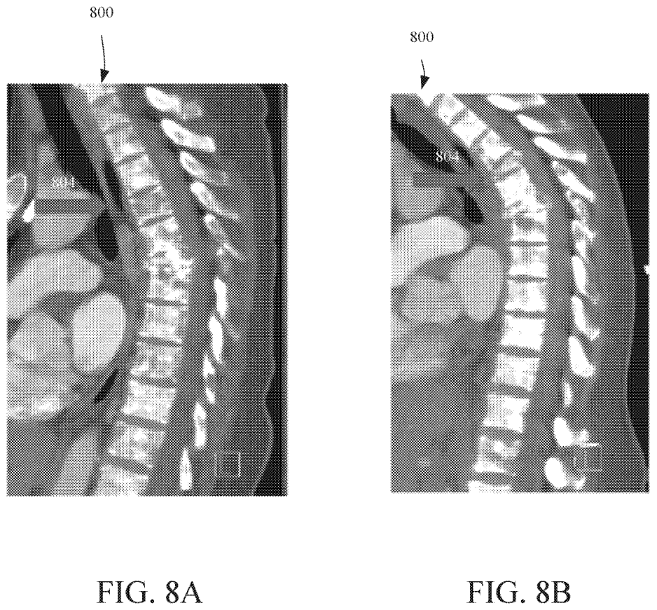

Referring now to FIGS. 8A-B, images of a vertebrae before and after fracture are presented. Referring to FIG. 8A, a spinal column 800 with vertebrae 804 is presented. Spinal column 800 has a preexisting thoracic level 6 compression fracture 804 (“preexisting fracture 804”). Spinal column 800 may be a tumor-infiltrated thoracolumbar spine. Spinal column 800 may have a plurality of vertebrae. For instance, spinal column 800 may have one or more vertebra 808. Vertebra 808 may be part of spinal column 800. Systems and methods described herein may identify vertebrae 804 as having a risk of future fracture. For instance, based on any radiomic features and/or clinical features described herein, a machine learning model may identify vertebrae 804 as having a high risk of future vertebrae fracture. Preexisting fracture 804 may increase a probability of a future risk of fracture of vertebra 808.

Referring now to FIG. 8B, spinal column 800 with vertebra 808 fractured is presented. Vertebra 808 may be located at thoracic level 4. Vertebra 808 may experience a vertebral compression fracture (VCF). Vertebra 808 may experience a burst fracture, as indicated by the retropulsion of the posterior wall of the vertebral body. In some embodiments, vertebra 808 may have become fractured within a day, week, month, year, or longer than a year since identification in FIG. 8A. Vertebra 808 may have become fractured due to one or more mechanical forces applied on vertebra 808 from preexisting fracture 804, forces applied by tumors, or other variables described herein.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

1. A computer-implemented method for predicting risks of vertebrae fracture in a patient, comprising:
obtaining image data of a patient;
identifying one or more vertebrae of the image data; and
analyzing, by a machine learning model, the one or more vertebrae to predict a future risk of vertebrae fracture of the one or more vertebrae for the patient, wherein the machine learning model is trained on a cohort of a population comprising at least one radiation naïve patient and wherein the analysis comprises processing features of the one or more images, the features comprising radiomic features that correlate to a structural integrity of one or more segments of the one or more vertebrae.

2. The method of claim 1, further comprising identifying a presence or absence of a fracture of the one or more vertebrae through a classifier.

3. The method of claim 2, wherein the machine learning model is trained to predict the future risk of vertebrae fracture based in part on the presence or absence of fracture of the one or more vertebrae.

4. The method of claim 1, wherein the radiomic features comprise one or more of shape-based, intensity-based, and texture-based features.

5. The method of claim 1, wherein the radiomic features comprise no more than top 20 selected radiomic features out of a plurality of radiomic features.

6. The method of claim 5, wherein the no more than top 20 selected radiomic features are identified through a feature selection process.

7. The method of claim 6, wherein the feature selection process is recursive feature elimination (RFE).

8. The method of claim 1, wherein the features comprise one or more clinical features.

9. The method of claim 1, wherein the one or more vertebrae are within vertebral levels from T1 through L5.

10. The method of claim 1, wherein the future risk of vertebrae fracture include a probability of vertebrae fracture of the one or more vertebrae within a one year timeline.

11. The method of claim 10, wherein the probability of vertebrae fracture is specific to a location within the one or more vertebrae.

12. The method of claim 1, wherein the image data comprises computerized tomography (CT) images.

13. The method of claim 12, wherein the CT images have a resolution of less than about 1920×1080.

14. The method of claim 12, where the CT images are of one or more of the chest, abdomen, and pelvis.

15. The method of claim 12, wherein the CT images comprise at least 75% of training data used to train the machine learning model.

16. The method of claim 1, further comprising generating a guided therapy based on the prediction of a future risk of vertebrae fracture.

17. An apparatus for predicting risks of fracture in a patient, comprising:
a processor; and
a memory communicatively connected to the processor, the memory containing instructions configuring the processor to:
obtain image data of a patient; and
execute a machine learning model using the image data as input, wherein the machine learning model is trained to:
identify one or more vertebrae of the image data, wherein the machine learning model is trained on a cohort of a population comprising at least one radiation naïve patient; and
analyze the one or more vertebrae to predict a future risk of vertebrae fracture of the one or more vertebrae for the patient, wherein in analyzing the one or more vertebra, the machine learning model is trained to process features of the one or more images, wherein the radiomic features correlate to a structural integrity of one or more segments of the one or more vertebrae.

18. The apparatus of claim 17, wherein the radiomic features comprise one or more of shape-based, intensity-based, and texture-based features.

19. The apparatus of claim 17, wherein the radiomic features comprise no more than top 20 selected radiomic features out of a plurality of radiomic features.

20. The apparatus of claim 19, wherein the no more than top 20 selected radiomic features are identified through a feature selection process.

21.-64. (canceled)
